SEQUENCE LISTING (37 sequences)

SEQ ID NO: 1 - DNA, 1038 bp, synthetic construct
  misc_feature 1..1038: note = ggt-UD
  source 1..1038: mol_type = other DNA
gcgcgctctagagtcgccgtttctcctctccggcagcatatgctgctgattcaacgaactatccggattacggttgctgtttaacatcccgccgttggtgttgggcagcatttgctgccgggcgggatttcgctcccccggctgtgactgcaacacccgctgagaattattgtttatctggttttctaaatgctgctgttgcaactgagtttgcgttttcagttgctgattcagcatccctttttgctggatttgctgagtctgcatctgggtttgcatccgctgctggctgggtatctgataccccggctggttagggttgttcagagtattaatgggctgtgcaaagccgacaaacggcaggagtgccgtaagaatcagaagtcgtttcatcgcgtatcctcctctgaagatatcctttaagtttactcgcttcccgacaaaacgatgattaattcagagttatataccaggcttagctggggttgccccttaatctctggagaataacgggttagcggccctcttcgtgggaagagggctattttgtcagggcaagccgaaggtagccttttttatttcgtaatcctgtagatattcttccagcggttttgccgttcccagagaatgaatttcaccactgtctttatcaataataaaaggtgcgttaccagctaagcgcgcggcctcatctccagtttcgagaaattctcgtgcttcgaaacagaaataccagccctggctaaagcgtccatgtagagtaatgacgaccgggagatctgcatcatcaaggtaatggttcgctttcgcgaatgcgtcgtgataagtaatcataattataataaatattattgttgagtgttatattattattcctgtgtgatacattgagcaaagacgcgttcatcttcgtcaaagatattattaaatgctggttttgaaaaaagttcatcggataagatatagtcacgaaataagtatctgctaatattttatgatttctttttcagaacgtcgaacgggattaagcttgcgcgc

SEQ ID NO: 2 - DNA, 1057 bp, synthetic construct
  misc_feature 1..1057: note = pepT-UD
  source 1..1057: mol_type = other DNA
gcgcgctctagaacgtgggtgatgtcctcgttatccagcatgatgcgtccggaatcaacagtttccagacctgcaatcaggcgaagaacggttgttttaccgcagccagaagggccaagcagcgtgaggaactcgccattgttgatagtcagatccagctggggaatgacctctttaccatcaaagcatttgcgaattcccgccaattgcaccagcggtgaaagcgaactcggttgtttattcaattttttactctgtcccatgtaaacgcaacggatggcttaccgatgcggggtttgtggttaaccaccttggtgactcttaatgagggcggtaattctacggcaaaccgcttgaatcgccaatctttgttgtgaattactggcttagctttatattcattaaggtaatgctgataaatattcccgcttgcaggggtaaaagtgacctgacgcaatatttgtcttttcttgcttcttaataatgttgtcacaaaaagtgagggtgactacgcgaaaagggatgcggcatgtgatgccgcatccggcttaaatccaaacttacccttcgaagaaccaatacccgctattgaccagcgccgcgagcatcgcgaggaatgacggatcttccagcgcatcgccaaaattctccgcagtcagcgcaatgttgctggcgagtgcatccagtgccggacggtgcggggaatcgatcttctcaccattggcatacacgtcgtcgccaatgcgcaatacgcgcagaccacccaggcgcaccagcacttcaccttgtttcagcgcatcgtagatttcatccggctgataaggcggctctggcggcgcgatatccagttcatgacgtgactgggatataaactcgccaaaccattgcttaaagtgttccggctggttgatcaattcgagcatcatctcacgcagtttatccatctcttgcggcagaacatccgcaggatgagcgcgaggtggaacatccggatcgctgtagtagttgccgcccagttcacgttgcagcacataatcggcaaatcaagcttgcgcgc

SEQ ID NO: 3 - DNA, 4130 bp, synthetic construct
  misc_feature 1..4130: note = pQEK1
  source 1..4130: mol_type = other DNA
gcttaaaggatcctaactcgagagatccctgccatttggcggggatttgctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagggtaccgaattccccgggctgcagactagtgagctccatatgcccaa

SEQ ID NO: 4 - DNA, 4230 bp, synthetic construct
  misc_feature 1..4230: note = pQEK1-term
  source 1..4230: mol_type = other DNA
gcttgactcctgttgatagatccagtaatgacctcagaactccatctggatttgttcagaacgctcggttgccgccgggcgttttttattggtgagaatagcttaaaggatcctaactcgagagatccctgccatttggcggggatttgctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagggtaccgaattccccgggctgcagactagtgagctccatatgcccaa

SEQ ID NO: 5 - DNA, 2691 bp, synthetic construct
  misc_feature 1..2691: note = PT5-ABTd(V260A) insert
  source 1..2691: mol_type = other DNA
cgggctgcagactagtaaatcataaaaaatttatttgctttgtgagcggataacaattataatagattcaattgtgagcggataacaatttcacacagaattcaaaagatctaagaaggagatatacatatatgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttatgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctgataaggtacctaaggaggttacaatgaaactgctgttcgtcgttgatcccctggccagcttgaaaccgtacaaggatagctccgttgccatgatgcgcgcagcgtgtgctcgtggtcatgccgtgttcgcagcagaagcgcgcgcactgctggttcgtgatggggtggctcgttctcgtgcagatgctgtcgaaacgcgtggcgacgatgactggtatcgcgttaccgaaacgcgtgaatttgccttaaccgactttgatgcagtggtgatgcgcgcagatccgcccgttgacgtggattaccttctcgcgacgcacctgttaggcgtagccgaaaccaacggtgcacgtgtcctgaatcgcccgcgtgccttgcgcgatttcaacgagaaactggccattctggaatttccgcagtttgtcgcacctaccctggtaagtgcggacgcaaccgaaattgcccactttctggctgctcatgcggatatcatcgtcaaaccgctgactgagatgggtggctccggtgtgtttcgcctgggagttagcgatccgaatcggaacgcgattctggagacattaacccgtcgtggctctcgcccaatcatggctcagcggtatttgccagcgatctcagagggcgacaaacgcatcctgctgatcgacggcgaagtagtgccatgggccttggcgcgcattccgctgaccggtgaaactcgcgggaatcttgcggctggtggtacagcgcgcgcgcaaccgctcagtgaacgggatcgcgaaatcgccgaaacgattgccccttgggcacgcagccagggcattttccttgcgggcttagacgtgattggggattgcctcaccgagattaacgtgacatcgcctactggatttcaggagattaccgcccaatcgggccatgatgttgcggaccagttcattgcggcgatcgaacgcgcgacgcgtccggaatgataacatatgcccaagcttgactcctgttgatagatc

SEQ ID NO: 6 - DNA, 6866 bp, synthetic construct
  misc_feature 1..6866: note = pQEK1-PT5-ABTd(V260A)-term
  source 1..6866: mol_type = other DNA
gcttgactcctgttgatagatccagtaatgacctcagaactccatctggatttgttcagaacgctcggttgccgccgggcgttttttattggtgagaatagcttaaaggatcctaactcgagagatccctgccatttggcggggatttgctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagggtaccgaatttcccgggctgcagactagtaaatcataaaaaatttatttgctttgtgagcggataacaattataatagattcaattgtgagcggataacaatttcacacagaattcaaaagatctaagaaggagatatacatatatgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttatgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctgataaggtacctaaggaggttacaatgaaactgctgttcgtcgttgatcccctggccagcttgaaaccgtacaaggatagctccgttgccatgatgcgcgcagcgtgtgctcgtggtcatgccgtgttcgcagcagaagcgcgcgcactgctggttcgtgatggggtggctcgttctcgtgcagatgctgtcgaaacgcgtggcgacgatgactggtatcgcgttaccgaaacgcgtgaatttgccttaaccgactttgatgcagtggtgatgcgcgcagatccgcccgttgacgtggattaccttctcgcgacgcacctgttaggcgtagccgaaaccaacggtgcacgtgtcctgaatcgcccgcgtgccttgcgcgatttcaacgagaaactggccattctggaatttccgcagtttgtcgcacctaccctggtaagtgcggacgcaaccgaaattgcccactttctggctgctcatgcggatatcatcgtcaaaccgctgactgagatgggtggctccggtgtgtttcgcctgggagttagcgatccgaatcggaacgcgattctggagacattaacccgtcgtggctctcgcccaatcatggctcagcggtatttgccagcgatctcagagggcgacaaacgcatcctgctgatcgacggcgaagtagtgccatgggccttggcgcgcattccgctgaccggtgaaactcgcgggaatcttgcggctggtggtacagcgcgcgcgcaaccgctcagtgaacgggatcgcgaaatcgccgaaacgattgccccttgggcacgcagccagggcattttccttgcgggcttagacgtgattggggattgcctcaccgagattaacgtgacatcgcctactggatttcaggagattaccgcccaatcgggccatgatgttgcggaccagttcattgcggcgatcgaacgcgcgacgcgtccggaatgataacatatgcccaa

SEQ ID NO: 7 - DNA, 1024 bp, synthetic construct
  misc_feature 1..1024: note = gor-UD
  source 1..1024: mol_type = other DNA
gcgcgctctagagtcgaaaaagccgacggtttccagcagcttaaggccaaactgccgccggtttcccgccgtggtttaatccttatcgacccgccgtatgaaatgaaaactgactatcaagcggtggtcagcgggatagcagaaggttacaaacgtttcgccactggtatttacgcactgtggtatccggtggtgctgcgtcagcaaattaagcgcatgatccacgatctggaagcgaccggtattcgcaaaattctgcaaattgaactggcggtactgccagacagcgatcgccgtggcatgaccgcttccggcatgattgtgattaacccgccgtggaaactggaacaacagatgaataacgtgctgccgtggctgcacagcaaactggttccggcaggcaccgggcacgccaccgtaagctggatcgtgccggagtaattgcagccattgctggcacctattacgtctcgcgctacaatcgcggtaatcaacgataaggacactttgtcatgttaaagggctaagagtagtgtgctcttagcccttaattacgtttccgctatcagttcagaagctgaagcagaaagcggatcagttccagcagcgcaattaacgcccctagaacgatgattgctttatcaatcacccgttttctccatgcgatggagtgagaatgcatccgcttactcatccactgcctgtcacggcgcatgtctcattgttagataagaactctctcactccggccagagcatcagttaacggcaccacccgtacttctgaccaggactttgaaagcgtttatgcgcattgccagagtgaaaatgcctcagagctaactggataatcatacagtacatgcaggttataaaaccagcacgtccttgcaatagtttcagtatggtattagcattgatgcgttagatgatggctatctcactccagtcagagccaccaactcagggctggaaagtaaaaaaccgacgcaaagtcggtttttttacatccgaagcttgcgcgc

SEQ ID NO: 8 - DNA, 2685 bp, synthetic construct
  misc_feature 1..2685: note = PT5-ABEc insert
  source 1..2685: mol_type = other DNA
cgggctgcagactagtaaatcataaaaaatttatttgctttgtgagcggataacaattataatagattcaattgtgagcggataacaatttcacacagaattcaaaagatctaagaaggagatatacatatatgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctgataaggtacctaaggaggttacaatgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaaaagcttgactcctgttgatagatc

SEQ ID NO: 9 - DNA, 6860 bp, synthetic construct
  misc_feature 1..6860: note = pQEK1-PT5-ABEc-term
  source 1..6860: mol_type = other DNA
gcttgactcctgttgatagatccagtaatgacctcagaactccatctggatttgttcagaacgctcggttgccgccgggcgttttttattggtgagaatagcttaaaggatcctaactcgagagatccctgccatttggcggggatttgctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagggtaccgaatttcccgggctgcagactagtaaatcataaaaaatttatttgctttgtgagcggataacaattataatagattcaattgtgagcggataacaatttcacacagaattcaaaagatctaagaaggagatatacatatatgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctgataaggtacctaaggaggttacaatgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaaaa

SEQ ID NO: 10 - DNA, 2408 bp, synthetic construct
  misc_feature 1..2408: note = PT5-FSa insert
  source 1..2408: mol_type = other DNA
cgggctgcagactagtaaatcataaaaaatttatttgctttgtgagcggataacaattataatagattcaattgtgagcggataacaatttcacacagaattcaaaagatctaagaaggagatatacatatatgattattgaccgtctgctgcaacgctcccatagccatctgccgatcctgcaagccacctttggtctggaacgtgaatccctgcgcattcatcagccgacccagcgtgtggcccagacgccgcatccgaaaaccctgggctctcgcaactatcacccgtacatccaaacggattatagtgaaccgcagctggaactgattaccccgatcgccaaagactctcaggaagcaatccgttttctgaaagccatttcagatgttgcaggtcgctcgattaatcatgacgaatatctgtggccgctgagtatgccgccaaaagtccgtgaagaagatattcaaatcgctcagctggaagatgcgttcgaatatgactaccgcaaatatctggaaaaaacctacggcaaactgattcagtccatctcaggtattcactataacctgggcctgggtcaagaactgctgacctcgctgtttgaactgagccaggcggataacgccattgacttccagaatcaactgtatatgaaactgtctcagaattttctgcgttaccgctggctgctgacctatctgtacggcgccagtccggtggcagaagaagatttcctggaccagaaactgaacaatccggtccgttccctgcgcaactcacatctgggttatgtgaatcacaaagatattcgtatctcgtataccagcctgaaagattacgtgaacgacctggaaaatgctgttaaatctggccagctgattgcggaaaaagaattttatagcccggttcgtctgcgcggctctaaagcctgccgtaactatctggaaaaaggtatcacgtacctggaatttcgcaccttcgatctgaatccgtttagtccgattggtatcacccaggaaacggtggataccgttcacctgtttctgctggcgctgctgtggattgacagctctagtcacatcgatcaggacattaaagaagccaaccgcctgaatgatctgatcgcactgagccatccgctggaaaaactgccgaaccaggctccggtttcagatctggtcgacgcaatgcaatcggtgattcagcactttaatctgagcccgtattaccaagatctgctggaaagtgttaaacgtcagatccaatccccggaactgaccgttgcgggtcagctgctggaaatgattgaaggtctgtccctggaaaccttcggccagcgccagggtcagatctatcatgattacgcatgggaagctccgtatgcgctgaaaggctacgaaacgatggaactgagcacccaactgctgctgttcgatgttattcagaaaggtgtgaactttgaagttctggatgaacaagaccagttcctgaaactgtggcataattcccacatcgaatatgtgaaaaacggcaatatgacgtcaaaagataactacatcgttccgctggcgatggccaataaagtggttaccaagaaaattctggacgaaaaacactttccgacgccgttcggtgatgaatttaccgaccgtaaagaagcgctgaactatttttcgcaaatccaggataaaccgattgtcgtgaaaccgaaaagcacgaacttcggcctgggtatttctatctttaaaaccagtgccaatctggcatcctacgaaaaagctattgatatcgcgtttacggaagacagcgcgatcctggtcgaagaatatattgaaggcaccgaataccgtttctttgtgctggagggtgattgtatcgcggtcctgctgcgtgtggccgcaaatgttgttggtgacggtatccataccatttcccaactggtgaaactgaaaaaccagaatccgctgcgtggctatgatcaccgctcaccgctggaagttatcgaactgggtgaagtcgaacagctgatgctggaacagcaaggctacacggtgaactcgatcccgccggaaggtaccaaaattgaactgcgtcgcaactctaatatcagtacgggcggtgatagcattgacgttaccaatacgatggatccgacctataaacagctggcagctgaaatggcagaagctatgggcgcatgggtctgtggtgtggatctgattatcccgaacgctacgcaggcgtacagcaaagacaagaaaaacgcgacctgcattgaactgaactttaatccgctgatgtatatgcatacctactgtcaagaaggtccgggtcagagcatcacgccgcgcatcctggcaaaactgtttccggaactgtaaaagcttgactcctgttgatagatc

SEQ ID NO: 11 - DNA, 6866 bp, synthetic construct; identical to SEQ ID NO: 6
  misc_feature 1..6866: note = pQEK1-PT5-FSa-term
  source 1..6866: mol_type = other DNA

SEQ ID NO: 12 - DNA, 1557 bp, Escherichia coli
  source 1..1557: mol_type = genomic DNA
atgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctga

SEQ ID NO: 13 - protein, 518 aa, Escherichia coli
MIPDVSQALAWLEKHPQALKGIQRGLERETLRVNADGTLATTGHPEALGSALTHKWITTDFAEALLEFITPVDGDIEHMLTFMRDLHRYTARNMGDERMWPLSMPCYIAEGQDIELAQYGTSNTGRFKTLYREGLKNRYGALMQTISGVHYNFSLPMAFWQAKCGDISGADAKEKISAGYFRVIRNYYRFGWVIPYLFGASPAICSSFLQGKPTSLPFEKTECGMYYLPYATSLRLSDLGYTNKSQSNLGITFNDLYEYVAGLKQAIKTPSEEYAKIGIEKDGKRLQINSNVLQIENELYAPIRPKRVTRSGESPSDALLRGGIEYIEVRSLDINPFSPIGVDEQQVRFLDLFMVWCALADAPEMSSSELACTRVNWNRVILEGRKPGLTLGIGCETAQFPLPQVGKDLFRDLKRVAQTLDSINGGEAYQKVCDELVACFDNPDLTFSARILRSMIDTGIGGTGKAFAEAYRNLLREEPLEILREEDFVAEREASERRQQEMEAADTEPFAVWLEKHA

SEQ ID NO: 14 - DNA, 951 bp, Escherichia coli
  source 1..951: mol_type = genomic DNA
atgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaa

SEQ ID NO: 15 - protein, 316 aa, Escherichia coli [sequence omitted]

SEQ ID NO: 16 - DNA, 945 bp, Thiobacillus denitrificans
  source 1..945: mol_type = genomic DNA
atgaagctgctgttcgtcgtcgatccgctggcgagcctcaagccgtacaaggacagttcggtcgcgatgatgcgcgcggcctgtgcgcgcggccacgccgtgttcgcggccgaagcacgcgcgctgctggtgcgcgacggagtcgcgcggtcgcgcgccgacgccgtcgagacgcgcggcgacgacgattggtatcgcgtgaccgaaacgcgcgaattcgcgctcacggatttcgatgccgttgtgatgcgcgccgacccgccggtcgacgtcgactacctgctcgcgacccacctgctcggcgtcgccgagaccaacggcgcgcgcgtgctgaaccggccgcgcgcgctgcgcgacttcaacgaaaaactcgccatcctcgagtttccgcaatttgtcgccccgacgctggtttcggccgacgcgacagaaatcgcccacttcctcgccgcccacgccgacatcatcgtcaagccgctcaccgagatgggcggcagcggcgtcttccgcctcggcgtttccgacccgaaccgcaacgccatcctcgaaacgctcacccgacgcggcagccggccgatcatggcgcagcgttatctgccggcgatcagcgaaggcgacaagcgcatcctgctgatcgacggcgaggtggtgccgtgggccctcgcgcggattccgctgacgggcgagacgcgcggcaatctcgccgcgggcggcacggcgcgtgcccagccgctttcggaacgcgaccgcgagatcgccgagacgatcgcgccctgggtgcgctcgcaaggcatcttcctcgccggcctcgacgtgatcggcgactgcctcaccgaaatcaacgtcacgagcccgaccggctttcaggaaatcacggcgcagagcggccacgacgtcgcggaccagttcatcgccgcgatcgagcgtgccacgcgtccggaataa

SEQ ID NO: 17 - protein, 314 aa, Thiobacillus denitrificans
MKLLFVVDPLASLKPYKDSSVAMMRAACARGHAVFAAEARALLVRDGVARSRADAVETRGDDDWYRVTETREFALTDFDAVVMRADPPVDVDYLLATHLLGVAETNGARVLNRPRALRDFNEKLAILEFPQFVAPTLVSADATEIAHFLAAHADIIVKPLTEMGGSGVFRLGVSDPNRNAILETLTRRGSRPIMAQRYLPAISEGDKRILLIDGEVVPWALARIPLTGETRGNLAAGGTARAQPLSERDREIAETIAPWVRSQGIFLAGLDVIGDCLTEINVTSPTGFQEITAQSGHDVADQFIAAIERATRPE

SEQ ID NO: 18 - DNA, 948 bp, synthetic construct
  misc_feature 1..948: note = TDgshB(V260A)
  source 1..948: mol_type = other DNA
atgaaactgctgttcgtcgttgatcccctggccagcttgaaaccgtacaaggatagctccgttgccatgatgcgcgcagcgtgtgctcgtggtcatgccgtgttcgcagcagaagcgcgcgcactgctggttcgtgatggggtggctcgttctcgtgcagatgctgtcgaaacgcgtggcgacgatgactggtatcgcgttaccgaaacgcgtgaatttgccttaaccgactttgatgcagtggtgatgcgcgcagatccgcccgttgacgtggattaccttctcgcgacgcacctgttaggcgtagccgaaaccaacggtgcacgtgtcctgaatcgcccgcgtgccttgcgcgatttcaacgagaaactggccattctggaatttccgcagtttgtcgcacctaccctggtaagtgcggacgcaaccgaaattgcccactttctggctgctcatgcggatatcatcgtcaaaccgctgactgagatgggtggctccggtgtgtttcgcctgggagttagcgatccgaatcggaacgcgattctggagacattaacccgtcgtggctctcgcccaatcatggctcagcggtatttgccagcgatctcagagggcgacaaacgcatcctgctgatcgacggcgaagtagtgccatgggccttggcgcgcattccgctgaccggtgaaactcgcgggaatcttgcggctggtggtacagcgcgcgcgcaaccgctcagtgaacgggatcgcgaaatcgccgaaacgattgccccttgggcacgcagccagggcattttccttgcgggcttagacgtgattggggattgcctcaccgagattaacgtgacatcgcctactggatttcaggagattaccgcccaatcgggccatgatgttgcggaccagttcattgcggcgatcgaacgcgcgacgcgtccggaatgataa

SEQ ID NO: 19 - DNA, 2253 bp, Streptococcus agalactiae
  source 1..2253: mol_type = genomic DNA
atgattattgaccgtctgctgcaacgctcccatagccatctgccgatcctgcaagccacctttggtctggaacgtgaatccctgcgcattcatcagccgacccagcgtgtggcccagacgccgcatccgaaaaccctgggctctcgcaactatcacccgtacatccaaacggattatagtgaaccgcagctggaactgattaccccgatcgccaaagactctcaggaagcaatccgttttctgaaagccatttcagatgttgcaggtcgctcgattaatcatgacgaatatctgtggccgctgagtatgccgccaaaagtccgtgaagaagatattcaaatcgctcagctggaagatgcgttcgaatatgactaccgcaaatatctggaaaaaacctacggcaaactgattcagtccatctcaggtattcactataacctgggcctgggtcaagaactgctgacctcgctgtttgaactgagccaggcggataacgccattgacttccagaatcaactgtatatgaaactgtctcagaattttctgcgttaccgctggctgctgacctatctgtacggcgccagtccggtggcagaagaagatttcctggaccagaaactgaacaatccggtccgttccctgcgcaactcacatctgggttatgtgaatcacaaagatattcgtatctcgtataccagcctgaaagattacgtgaacgacctggaaaatgctgttaaatctggccagctgattgcggaaaaagaattttatagcccggttcgtctgcgcggctctaaagcctgccgtaactatctggaaaaaggtatcacgtacctggaatttcgcaccttcgatctgaatccgtttagtccgattggtatcacccaggaaacggtggataccgttcacctgtttctgctggcgctgctgtggattgacagctctagtcacatcgatcaggacattaaagaagccaaccgcctgaatgatctgatcgcactgagccatccgctggaaaaactgccgaaccaggctccggtttcagatctggtcgacgcaatgcaatcggtgattcagcactttaatctgagcccgtattaccaagatctgctggaaagtgttaaacgtcagatccaatccccggaactgaccgttgcgggtcagctgctggaaatgattgaaggtctgtccctggaaaccttcggccagcgccagggtcagatctatcatgattacgcatgggaagctccgtatgcgctgaaaggctacgaaacgatggaactgagcacccaactgctgctgttcgatgttattcagaaaggtgtgaactttgaagttctggatgaacaagaccagttcctgaaactgtggcataattcccacatcgaatatgtgaaaaacggcaatatgacgtcaaaagataactacatcgttccgctggcgatggccaataaagtggttaccaagaaaattctggacgaaaaacactttccgacgccgttcggtgatgaatttaccgaccgtaaagaagcgctgaactatttttcgcaaatccaggataaaccgattgtcgtgaaaccgaaaagcacgaacttcggcctgggtatttctatctttaaaaccagtgccaatctggcatcctacgaaaaagctattgatatcgcgtttacggaagacagcgcgatcctggtcgaagaatatattgaaggcaccgaataccgtttctttgtgctggagggtgattgtatcgcggtcctgctgcgtgtggccgcaaatgttgttggtgacggtatccataccatttcccaactggtgaaactgaaaaaccagaatccgctgcgtggctatgatcaccgctcaccgctggaagttatcgaactgggtgaagtcgaacagctgatgctggaacagcaaggctacacggtgaactcgatcccgccggaaggtaccaaaattgaactgcgtcgcaactctaatatcagtacgggcggtgatagcattgacgttaccaatacgatggatccgacctataaacagctggcagctgaaatggcagaagctatgggcgcatgggtctgtggtgtggatctgattatcccgaacgctacgcaggcgtacagcaaagacaagaaaaacgcgacctgcattgaactgaactttaatccgctgatgtatatgcatacctactgtcaagaaggtccgggtcagagcatcacgccgcgcatcctggcaaaactgtttccggaactgtaa

SEQ ID NO: 20 - protein, 750 aa, Streptococcus agalactiae
MIIDRLLQRSHSHLPILQATFGLERESLRIHQPTQRVAQTPHPKTLGSRNYHPYIQTDYSEPQLELITPIAKDSQEAIRFLKAISDVAGRSINHDEYLWPLSMPPKVREEDIQIAQLEDAFEYDYRKYLEKTYGKLIQSISGIHYNLGLGQELLTSLFELSQADNAIDFQNQLYMKLSQNFLRYRWLLTYLYGASPVAEEDFLDQKLNNPVRSLRNSHLGYVNHKDIRISYTSLKDYVNDLENAVKSGQLIAEKEFYSPVRLRGSKACRNYLEKGITYLEFRTFDLNPFSPIGITQETVDTVHLFLLALLWIDSSSHIDQDIKEANRLNDLIALSHPLEKLPNQAPVSDLVDAMQSVIQHFNLSPYYQDLLESVKRQIQSPELTVAGQLLEMIEGLSLETFGQRQGQIYHDYAWEAPYALKGYETMELSTQLLLFDVIQKGVNFEVLDEQDQFLKLWHNSHIEYVKNGNMTSKDNYIVPLAMANKVVTKKILDEKHFPTPFGDEFTDRKEALNYFSQIQDKPIVVKPKSTNFGLGISIFKTSANLASYEKAIDIAFTEDSAILVEEYIEGTEYRFFVLEGDCIAVLLRVAANVVGDGIHTISQLVKLKNQNPLRGYDHRSPLEVIELGEVEQLMLEQQGYTVNSIPPEGTKIELRRNSNISTGGDSIDVTNTMDPTYKQLAAEMAEAMGAWVCGVDLIIPNATQAYSKDKKNATCIELNFNPLMYMHTYCQEGPGQSITPRILAKLFPEL

SEQ ID NO: 21 - DNA, 1743 bp, Escherichia coli
  source 1..1743: mol_type = genomic DNA
atgataaaaccgacgtttttacgccgggtggccattgctgctctgctctcaggaagttgttttagcgccgccgccgcgcctcctgcgccgcccgtctcgtatggtgtggaggaagatgtcttccacccggtacgcgcgaaacagggaatggtagcgtctgtggacgccactgccactcaggtgggggtggatattctcaaggagggcgggaatgccgttgatgccgccgtggcggtgggctacgcgctggcggtaacgcatccgcaggcagggaatctgggcggtggtggttttatgttaatccgctcgaaaaatggcaataccacggctatcgatttccgcgaaatggcacccgccaaagcgacccgcgatatgttcctcgatgatcagggcaacccggacagcaaaaaatcactcacttcgcatctggcttccggcacaccgggtacggtagcaggtttctcgctggcgctggataaatacggcaccatgccgctgaacaaagtcgtgcagcccgcgtttaaactggcacgcgatggttttatcgttaacgacgcgctggctgacgatctcaaaacctacggtagcgaagtgttgccgaatcacgaaaacagtaaagctatcttctggaaagagggcgagccgctgaaaaagggcgacacgctggtgcaggcgaacctggcaaagagcctggagatgattgctgaaaacggcccggacgaattctataaaggcacgattgcggaacagatcgcccaggagatgcagaaaaacggtggcttgatcactaaagaagatttagcagcctataaagcggtcgaacgcactccgataagcggcgattatcgcgggtatcaggtttactccatgccaccgccatcctccggcgggatccatatcgtacaaatcctcaatattctggaaaacttcgatatgaagaaatacggctttggcagcgccgatgcgatgcaaatcatggcagaagcggagaaatacgcctacgccgaccgctcggaatatcttggcgacccggattttgtcaaagtaccgtggcaggcgctgaccaataaagcctatgccaaatctattgccgatcaaattgatatcaataaagcgaagccatccagcgaaattcgccccggcaagcttgcgccttatgagagtaatcaaactacccattactcagtggtggataaagatggtaacgcggtggcggtgacctatacgctgaacaccaccttcggtacgggcattgtcgcgggcgagagcggtattctgcttaataaccagatggatgatttctccgccaaaccgggcgtaccgaacgtttacgggctggtgggcggtgatgccaacgccgtcgggccgaacaaacgcccgctgtcgtcgatgtcgccgaccattgtggtgaaagacggtaaaacctggctggttaccggtagcccaggcggtagccggatcatcactacagtgctgcaaatggtggtgaatagcatcgattatggcttgaacgtcgccgaagcgaccaatgcgccgcgtttccaccatcagtggttgccggacgagctgcgtgtcgaaaaagggtttagcccggatacgctcaagctgctggaagcaaaaggtcagaaagtggcgctgaaagaggcgatgggcagtacacaaagcattatggttgggccggacggtgagttgtacggcgcatccgacccgcgctcggtggatgatttaacggcggggtactaa

SEQ ID NO: 22 - protein, 580 aa, Escherichia coli [sequence omitted]

SEQ ID NO: 23 - DNA, 1227 bp, Escherichia coli
  source 1..1227: mol_type = genomic DNA
atggataaactacttgagcgatttttgaactacgtgtctctggatacccaatcaaaagcaggggtgagacaggttcccagcacggaaggccaatggaagttattgcatctgctgaaagagcagctcgaagagatggggcttatcaatgtgaccttaagtgagaagggcactttgatggcgacgttaccggctaacgtccctggcgatatcccggcgattggctttatttctcatgtggatacctcaccggattgcagcggcaaaaatgtgaatccgcaaattgttgaaaactatcgcggtggcgatattgcgctgggtatcggcgatgaagttttatcaccggttatgttcccggtgctgcatcagctactgggtcagacgctgattaccaccgatggtaaaaccttgttaggtgccgatgacaaagcaggtattgcagaaatcatgaccgcgctggcggtattgcaacagaaaaaaattccgcatggtgatattcgcgtcgcctttaccccggatgaagaagtgggcaaaggggcgaaacattttgatgttgacgccttcgatgcccgctgggcttacactgttgatggtggtggcgtaggcgaactggagtttgaaaacttcaacgccgcgtcggtcaatatcaaaattgtcggtaacaatgttcatccgggcacggcgaaaggagtgatggtaaatgcgctgtcgctggcggcacgtattcatgcggaagttccggcggatgaaagcccggaaatgacagaaggctatgaaggtttctatcatctggcgagcatgaaaggcaccgttgaacgggccgatatgcactacatcatccgtgatttcgaccgtaaacagtttgaagcgcgtaaacgtaaaatgatggagatcgccaaaaaagtgggcaaagggttacatcctgattgctacattgaactggtgattgaagacagttactacaatatgcgcgagaaagtggttgagcatccgcatattctcgatatcgcccagcaggcgatgcgcgattgcgatattgaaccggaactgaaaccgatccgcggtggtaccgacggcgcgcagttgtcgtttatgggattaccgtgcccgaacctgttcactggcggttacaactatcatggtaagcatgagtttgtgactctggaaggtatggaaaaagcggtgcaggtgatcgtccgtattgccgagttaacggcgcaacggaagtaa

SEQ ID NO: 24 - protein, 408 aa, Escherichia coli
MDKLLERFLNYVSLDTQSKAGVRQVPSTEGQWKLLHLLKEQLEEMGLINVTLSEKGTLMATLPANVPGDIPAIGFISHVDTSPDCSGKNVNPQIVENYRGGDIALGIGDEVLSPVMFPVLHQLLGQTLITTDGKTLLGADDKAGIAEIMTALAVLQQKKIPHGDIRVAFTPDEEVGKGAKHFDVDAFDARWAYTVDGGGVGELEFENFNAASVNIKIVGNNVHPGTAKGVMVNALSLAARIHAEVPADESPEMTEGYEGFYHLASMKGTVERADMHYIIRDFDRKQFEARKRKMMEIAKKVGKGLHPDCYIELVIEDSYYNMREKVVEHPHILDIAQQAMRDCDIEPELKPIRGGTDGAQLSFMGLPCPNLFTGGYNYHGKHEFVTLEGMEKAVQVIVRIAELTAQRK

SEQ ID NO: 25 - DNA, 1353 bp, Escherichia coli
  source 1..1353: mol_type = genomic DNA
atgactaaacactatgattacatcgccatcggcggcggcagcggcggtatcgcctccatcaaccgcgcggctatgtacggccagaaatgtgcgctgattgaagccaaagagctgggcggcacctgcgtaaatgttggctgtgtgccgaaaaaagtgatgtggcacgcggcgcaaatccgtgaagcgatccatatgtacggcccggattatggttttgataccactatcaataaattcaactgggaaacgttgatcgccagccgtaccgcctatatcgaccgtattcatacttcctatgaaaacgtgctcggtaaaaataacgttgatgtaatcaaaggctttgcccgcttcgttgatgccaaaacgctggaggtaaacggcgaaaccatcacggccgatcatattctgatcgccacaggcggtcgtccgagccacccggatattccgggcgtggaatacggtattgattctgatggcttcttcgcccttcctgctttgccagagcgcgtggcggttgttggcgcgggttacatcgccgttgagctggcgggcgtgattaacggcctcggcgcgaaaacgcatctgtttgtgcgtaaacatgcgccgctgcgcagcttcgacccgatgatttccgaaacgctggtcgaagtgatgaacgccgaaggcccgcagctgcacaccaacgccatcccgaaagcggtagtgaaaaataccgatggtagcctgacgctggagctggaagatggtcgcagtgaaacggtggattgcctgatttgggcgattggtcgcgagcctgccaatgacaacatcaacctggaagccgctggcgttaaaactaacgaaaaaggctatatcgtcgtcgataaatatcaaaacaccaatattgaaggtatttacgcggtgggcgataacacgggtgcagtggagctgacaccggtggcagttgcagcgggtcgccgtctctctgaacgcctgtttaataacaagccggatgagcatctggattacagcaacattccgaccgtggtcttcagccatccgccgattggtactgttggtttaacggaaccgcaggcgcgcgagcagtatggcgacgatcaggtgaaagtgtataaatcctctttcaccgcgatgtataccgccgtcaccactcaccgccagccgtgccgcatgaagctggtgtgcgttggatcggaagagaagattgtcggtattcacggcattggctttggtatggacgaaatgttgcagggcttcgcggtggcgctgaagatgggggcaaccaaaaaagacttcgacaataccgtcgccattcacccaacggcggcagaagagttcgtgacaatgcgttaa

SEQ ID NO: 26 - protein, 450 aa, Escherichia coli
MTKHYDYIAIGGGSGGIASINRAAMYGQKCALIEAKELGGTCVNVGCVPKKVMWHAAQIREAIHMYGPDYGFDTTINKFNWETLIASRTAYIDRIHTSYENVLGKNNVDVIKGFARFVDAKTLEVNGETITADHILIATGGRPSHPDIPGVEYGIDSDGFFALPALPERVAVVGAGYIAVELAGVINGLGAKTHLFVRKHAPLRSFDPMISETLVEVMNAEGPQLHTNAIPKAVVKNTDGSLTLELEDGRSETVDCLIWAIGREPANDNINLEAAGVKTNEKGYIVVDKYQNTNIEGIYAVGDNTGAVELTPVAVAAGRRLSERLFNNKPDEHLDYSNIPTVVFSHPPIGTVGLTEPQAREQYGDDQVKVYKSSFTAMYTAVTTHRQPCRMKLVCVGSEEKIVGIHGIGFGMDEMLQGFAVALKMGATKKDFDNTVAIHPTAAEEFVTMR

SEQ ID NO: 27 - DNA, 1140 bp, Escherichia coli
  source 1..1140: mol_type = genomic DNA
atggttttgcaatccacgcgctggttggcgctcggctatttcacatacttttttagttacggcatttttctacctttctggagcgtctggcttaaagggattggtttaacgccagaaaccatcggcctgttattgggggcaggtctggttgcccgtttcctcgggagtttgctcatcgcgccccgcgtcagcgatccttcccgcctgatttccgccttgcgcgtgctggcactgctgacacttctctttgctgtcgccttctgggcgggggcgcacgtagcgtggctgatgctggtgatgattggctttaacctctttttctcaccactggtaccgttgaccgatgcactggcgaatacgtggcaaaagcagttcccgcttgattacggcaaagtgcgactgtggggctcggtggcgtttgtcattggctcggcgctgacgggcaaactggtcactatgtttgattatcgggtgatcctcgcgctgttgacgttgggcgtggcatccatgctgctcggctttctcatccgtccgacgattcagccacaaggggcaagtcgccagcaggagagcaccggttggtctgcgtggttggcgctggttcgccagaactggcgctttctggcctgcgtttgtttattgcagggggcacatgcggcctattacggttttagcgccatttactggcaggcagctggctactcggcctcggcggtggggtatttgtggtcgctgggcgtggtggcggaagtcattatctttgcgctgagtaataaacttttccgccgttgtagtgcacgcgatatgctgttgatctcggcgatttgcggcgtagtgcgctggggcattatgggagcaactacggcgttgccgtggttgatagtggtgcaaattctgcattgcggcaccttcacggtctgccacctggccgccatgcgttatattgctgctcgccagggtagcgaagtcatccgtttacaggcggtttactctgccgtcgcgatgggcggcagtatcgctatcatgaccgttttcgccggtttcctgtatcaatatctgggccacggcgtgttctgggtaatggcgctggtggcgcttccggcaatgtttttgcgcccgaaagttgttccctcatgctga

SEQ ID NO: 28 - DNA, 20 bp, synthetic construct
  misc_feature 1..20: note = hcaT forward primer
  source 1..20: mol_type = other DNA
gctgctcggctttctcatcc

SEQ ID NO: 29 - DNA, 19 bp, synthetic construct
  misc_feature 1..19: note = hcaT reverse primer
  source 1..19: mol_type = other DNA
ccaaccacgcagaccaacc

SEQ ID NO: 30 - DNA, 20 bp, synthetic construct
  misc_feature 1..20: note = gshA forward primer
  source 1..20: mol_type = other DNA
ctggctggaaaaacatcctc

SEQ ID NO: 31 - DNA, 20 bp, synthetic construct
  misc_feature 1..20: note = gshA reverse primer
  source 1..20: mol_type = other DNA
tcagtgcggaacctaatgct

SEQ ID NO: 32 - DNA, 18 bp, synthetic construct
  misc_feature 1..18: note = TDgshB(V260A) forward primer
  source 1..18: mol_type = other DNA
tttccttgcgggcttaga

SEQ ID NO: 33 - DNA, 20 bp, synthetic construct
  misc_feature 1..20: note = TDgshB(V260A) reverse primer
  source 1..20: mol_type = other DNA
attgggcggtaatctcctga

SEQ ID NO: 34 - DNA, 20 bp, synthetic construct
  misc_feature 1..20: note = gshB forward primer
  source 1..20: mol_type = other DNA
tgtttaccgcctggttctct

SEQ ID NO: 35 - DNA, 20 bp, synthetic construct
  misc_feature 1..20: note = gshB reverse primer
  source 1..20: mol_type = other DNA
tgatgtcgctgtgtttctcc

SEQ ID NO: 36 - DNA, 20 bp, synthetic construct
  misc_feature 1..20: note = SAgshF forward primer
  source 1..20: mol_type = other DNA
cggataacgccattgacttc

SEQ ID NO: 37 - DNA, 21 bp, synthetic construct
  misc_feature 1..21: note = SAgshF reverse primer
  source 1..21: mol_type = other DNA
cggattgttcagtttctggtc

(19) United States
(12) Patent Application Publication
(10) Pub. No.: US 2025/0382653 A1
(43) Pub. Date: Dec. 18, 2025

(54) METHOD FOR PRODUCING USEFUL SUBSTANCE

(71) Applicant: KANEKA CORPORATION, Osaka-shi, Osaka (JP)

(72) Inventors: Misato MATSUI, Takasago-shi (JP); Shingo KOBAYASHI, Takasago-shi (JP); Naoaki TAOKA, Takasago-shi (JP)

(73) Assignee: KANEKA CORPORATION, Osaka-shi, Osaka (JP)

(21) Appl. No.: 19/302,863

(22) Filed: Aug. 18, 2025

Related U.S. Application Data
(62) Division of application No. 17/749,857, filed on May 20, 2022.
Application No. 17/749,857 is a continuation of application No. PCT/JP2020/043356, filed on Nov. 20, 2020.

(30) Foreign Application Priority Data
Nov. 22, 2019 (JP) 2019-211477
Jan. 9, 2020 (JP) 2020-002363

(51) Int. Cl. C12P 21/02; C12N 9/00; C12N 9/02; C12N 9/10; C12N 9/48; C12P 13/00

(52) U.S. Cl. CPC C12P 21/02; C12N 9/0051; C12N 9/104; C12N 9/485; C12N 9/93; C12P 13/005; C12Y 108/01007; C12Y 203/02002; C12Y 304/11004; C12Y 603/02002; C12Y 603/02003

(57) ABSTRACT

The present disclosure concerns a method for producing peptides such as glutathione and a microorganism that can be used for such method. One or more embodiments of the first aspect of the present disclosure concern a method for producing peptides such as glutathione comprising culturing a prokaryotic microbial strain in which the expression levels of one or more genes selected from among the gshA gene, the gshB gene, and the gshF gene are enhanced, compared with the expression levels thereof in the wild-type strain thereof in a medium in which the total concentration of cysteine and cystine is 0.5 g/l or lower. The second aspect of the present disclosure concerns a microorganism comprising disruptions of the γ-glutamyltransferase gene and the glutathione reductase gene and exhibiting the enhanced expression levels of the gshA gene and the gshB or gshF gene.

### CROSS-REFERENCE TO RELATED APPLICATIONS

This application is a divisional of U.S. application Ser. No. 17/749,857 filed May 20, 2022, which is a Continuation of PCT International Application No. PCT/JP2020/043356, filed on Nov. 20, 2020, which claims priority under 35 U.S.C. § 119(a) to Japanese Patent Application No. 2019-211477 filed Nov. 22, 2019, and Japanese Patent Application No. 2020-002363 filed Jan. 9, 2020, all of which are hereby expressly incorporated by reference into the present application.

### REFERENCE TO ELECTRONIC SEQUENCE LISTING

This application contains a Sequence Listing which has been submitted electronically in .xml format and is hereby incorporated by reference in its entirety. Said.xml copy, created on Aug. 12, 2025, is named “PH-8539-PCT.xml” and is 96,813 bytes in size. The sequence listing contained in this.xml file is part of the specification and is hereby incorporated by reference herein in its entirety.

### TECHNICAL FIELD

One or more embodiments of the first aspect of the present disclosure relates to a method for producing γ-glutamylcysteine, bis-γ-glutamylcystine, γ-glutamylcystine, reduced glutathione, and/or oxidized glutathione.

Other one or more embodiments of the first aspect of the present disclosure relates to a prokaryotic microbial strain capable of overproduction of γ-glutamylcysteine, bis-γ-glutamylcystine, γ-glutamylcystine, reduced glutathione, and/or oxidized glutathione.

The second aspect of the present disclosure relates to a microorganism that produces glutathione, comprising a disruption of the glutathione reductase gene and a method for producing glutathione using the microorganism.

### BACKGROUND ART

A glutathione reduced form and a glutathione oxidized form are known to exist, and reduced glutathione is a peptide comprising 3 amino acids; i.e., L-cysteine, L-glutamic acid, and glycine. Oxidized glutathione is a compound resulting from a disulfide bond formed between thiol groups of bimolecular reduced glutathione. Oxidized glutathione is a compound that exists in a wide variety of organisms, such as animals including humans, plants, and microorganisms, and is important for organisms that play a role in, for example, elimination of reactive oxygen species, detoxication, and amino acid metabolism. Accordingly, oxidized glutathione has drawn attention in the fields of pharmaceutical, food, and cosmetic industries. In recent years, oxidized glutathione was found to have effects of accelerating plant growth and other effects. Accordingly, use of oxidized glutathione is expected in a wide variety of fields, including the agricultural field.

Glutathione is present in an organism in either of a glutathione reduced form in which the thiol group of L-cysteine is in a reduced SH form (hereafter, it may be referred to as “GSH”) or a glutathione oxidized form in which the thiol groups of L-cysteine are oxidized to form a disulfide bond between 2 glutathione molecules (hereafter, it may be referred to as “GSSG”).

Examples of known methods for producing glutathione include a method for producing glutathione by fermentation using yeast or Escherichia coli (Patent Document 1) and a method for producing glutathione comprising producing γ-glutamylcysteine synthetase or glutathione synthetase using microorganisms and enzymatically ligating L-glutamic acid, L-cysteine, and glycine (Patent Documents 3 and 4).

For example, Patent Document 1 discloses a method for producing glutathione comprising culturing yeast strains with increased thiol oxidase activity compared with that of parent strains in a medium to produce glutathione and collecting glutathione from the resulting culture solution.

Patent Document 2 discloses a method for producing glutathione or γ-glutamylcysteine comprising culturing microorganisms with activity of proteins having glutathione transport activity and activity of proteins associated with biosynthesis of glutathione or γ-glutamylcysteine higher than those of parent strains in a medium to produce and accumulate glutathione or 65 -glutamylcysteine in the medium and collecting glutathione or γ-glutamylcysteine from the culture product. Patent Document 2 describes, in Example 4, that E. coli strains overexpressing theE. coli-derived glutamate-cysteine ligase gshA gene and the glutathione synthetase gshB gene were cultured in an amino-acid-supplemented medium and the glutathione concentration in the medium was 160 mg/l.

Non-Patent Document 1 describes a method for producing glutathione comprising culturing E. coli strains transformed with an expression vector comprising the bifunctional glutathione synthetase gshF gene under the control of a constitutive promoter in a medium supplemented with constituent amino acids of glutathione; i.e., L-cysteine, L-glutamic acid, and glycine.

### PRIOR ART DOCUMENTS

### Patent Documents

Patent Document 1: WO 2016/140349

Patent Document 2: WO 2008/126784

Patent Document 3: JP S60-27396 A (1985)

Patent Document 4: JP S60-27397 A (1985)

### Non-Patent Documents

Non-Patent Document 1: Journal of Biotechnology, 2018, ht

doi.org/10.1016/j.jbiotec.2018.11.001

### SUMMARY OF THE INVENTION

### Objects to Be Attained by the Invention

Firstly, novel embodiments of a prokaryotic microbial strain capable of producing γ-glutamylcysteine, bis-γ-glutamylcystine, γ-glutamylcystine, reduced glutathione, and/or oxidized glutathione and a method for producing the peptide using the strain are desired.

Secondly, novel embodiments of a microorganism capable of producing glutathione and a method for producing glutathione using the microorganism are desired.

### Means for Attaining the Objects

The first aspect of the present disclosure includes the embodiments described in (1) to (14) below.
(1) A method for producing γ-glutamylcysteine, bis-γ-glutamylcystine, γ-glutamylcystine, reduced glutathione, and/or oxidized glutathione, comprising
   culturing a prokaryotic microbial strain in a medium in which the total concentration of cysteine and cystine is 0.5 g/l or lower, the prokaryotic microbial strain exhibiting expression levels of one or more genes selected from genes encoding a glutamate-cysteine ligase, a glutathione synthetase, and a bifunctional glutathione synthetase increased compared with those in the wild-type strain thereof.
(2) The method according to (1), wherein the prokaryotic microbial strain is capable of overproduction of γ-glutamylcysteine, bis-γ-glutamylcystine, γ-glutamylcystine, reduced glutathione, and/or oxidized glutathione by induced expression of the one or more genes.
(3) A method for producing γ-glutamylcysteine, bis-γ-glutamylcystine, γ-glutamylcystine, reduced glutathione, and/or oxidized glutathione, comprising
   culturing a prokaryotic microbial strain in a medium, the prokaryotic microbial strain exhibiting expression levels of one or more genes selected from genes encoding a glutamate-cysteine ligase, a glutathione synthetase, and a bifunctional glutathione synthetase, increased compared with those in the wild-type strain thereof, and
   not comprising adding cysteine or cystine to the medium.
(4) The method according to (3), wherein the prokaryotic microbial strain is capable of overproducing γ-glutamylcysteine, bis-γ-glutamylcystine, γ-glutamylcystine, reduced glutathione, and/or oxidized glutathione by induced expression of the one or more genes.
(5) A prokaryotic microbial strain capable of overproducing γ-glutamylcysteine, bis-γ-glutamylcystine, γ-glutamylcystine, reduced glutathione, and/or oxidized glutathione, which carry one or more genes selected from genes encoding a glutamate-cysteine ligase, aglutathione synthetase, and a bifunctional glutathione synthetase, operably linked to a promoter
   wherein, when the one or more genes is the glutamate-cysteine ligase gene, the promoter increases the amount of transcription of the glutamate-cysteine ligase gene in the prokaryotic microbial strain by at least 20 times greater than that of the wild-type strain thereof.
(6) The prokaryotic microbial strain according to (5), wherein the promoter is an inducible promoter.
(7) The prokaryotic microbial strain according to (6), wherein the inducible promoter is IPTG inducible promoter, photoinducible promoter, araBAD promoter, rhaBAD promoter, tet promoter, penP promoter, cspA promoter, or a promoter comprising, as an operator sequence, tetO or lacO operator.
(8) The prokaryotic microbial strain according to (7), wherein the inducible promoter is T5 promoter, T7 promoter, lacT5 promoter, lacT7 promoter, tac promoter, araBAD promoter, rhaBAD promoter, tet promoter, penP promoter, cspA promoter, or a promoter comprising, as an operator sequence, tetO or lacO operator.
(9) The prokaryotic microbial strain according to (8), wherein the inducible promoter is T5 promoter, T7 promoter, lacT5 promoter, lacT7 promoter, or tac promoter.
(10) The prokaryotic microbial strain according to (9), wherein the inducible promoter is T5 promoter.
(11) The prokaryotic microbial strain according to any of (5) to (10), which is a transformed enteric bacterium.
(12) The prokaryotic microbial strain according to any of (5) to (10), which is a transformed E. coli strain.
(13) The method according to (1) or (2), wherein the prokaryotic microbial strain is the prokaryotic microbial strain according to any of (5) to (12).
(14) The method according to (3) or (4), wherein the prokaryotic microbial strain is the prokaryotic microbial strain according to any of (5) to (12).

The second aspect of the present disclosure includes the embodiments described in (15) to (22) below.
(15) A microorganism comprising disruptions of the gene [1] and the gene [2] below and exhibiting enhanced expression levels of the genes [3] or the gene [4] below:
   [1] a gene encoding γ-glutamyltransferase (EC: 2.3.2.2);
   [2] a gene encoding glutathione reductase (EC: 1.8.1.7);
   [3] a gene encoding glutamate-cysteine ligase (EC: 6.3.2.2) and a gene encoding glutathione synthetase (EC: 6.3.2.3); and
   [4] a gene encoding bifunctional glutathione synthetase.
(16) The microorganism according to (15), comprising a disruption of the gene [5] below:
   [5] a gene encoding tripeptide peptidase (EC: 3.4.11.4).
(17) The microorganism according to (15) or (16), wherein the microorganism is a transformed bacterium.
(18) The microorganism according to (15) or (16), wherein the microorganism is a transformed enteric bacterium.
(19) The microorganism according to (15) or (16), wherein the microorganism is a transformed Gram-negative bacterium.
(20) The microorganism according to (15) or (16), wherein the microorganism is a transformed E. coli strain.
(21) A method for producing glutathione comprising culturing the microorganism according to any of (15) to (20) in a medium.

This description includes a part, or all of the contents as disclosed in the descriptions and/or drawings of Japanese Patent Application Nos. 2019-211477 and 2020-002363, which are priority documents of the present disclosure.

### Effects of the Invention

According to the method of the first aspect of the present disclosure, a step of adding cysteine or cystine is not required. Thus, γ-glutamylcysteine, bis-γ-glutamylcystine, γ-glutamylcystine, reduced glutathione, and/or oxidized glutathione can be produced at low cost.

The prokaryotic microbial strain according to the first aspect of the present disclosure is capable of efficient production of γ-glutamylcysteine, bis-γ-glutamylcystine, γ-glutamylcystine, reduced glutathione, and/or oxidized glutathione.

The microorganism according to the second aspect of the present disclosure can yield high glutathione productivity by fermentation.

The method for producing glutathione according to the second aspect of the present disclosure enables efficient production of glutathione.

The third aspect of the present disclosure includes the embodiments described in (1) to (15) below.
(1) A method for producing γ-glutamylcysteine, bis-γ-glutamylcystine, γ-glutamylcystine, reduced glutathione, and/or oxidized glutathione, comprising
   culturing a gram-negative bacterium in a medium, wherein the total concentration of cysteine and cystine in the medium is 0.5 g/l or lower before inoculation of the gram-negative bacterium, thereby increasing the expression levels of one or more genes selected from genes encoding glutamate-cysteine ligase, glutathione synthetase, and bifunctional glutathione synthetase, when compared with expression levels in a wild-type strain thereof, wherein the gram negative bacterium is capable of overproducing γ-glutamylcysteine, bis-γ-glutamylcystine, γ-glutamylcystine, reduced glutathione, and/or oxidized glutathione by induced expression of the one or more genes when cultured in medium.
(2) The method according to (1), wherein the gram-negative bacterium carries one or more genes selected from genes encoding glutamate-cysteine ligase, glutathione synthetase, and bifunctional glutathione synthetase, operably linked to an inducible promoter,
   wherein, when the one or more genes is the gene encoding glutamate-cysteine ligase, the inducible promoter increases the expression level of the gene encoding glutamate-cysteine ligase in the gram-negative bacterium by at least 20 times greater than that of the wild-type strain thereof.
(3) The method according to (2), wherein the inducible promoter is IPTG inducible promoter, photoinducible promoter, araBAD promoter, rhaBAD promoter, tet promoter, penP promoter, cspA promoter, or a promoter comprising, as an operator sequence, tetO or lacO operator.
(4) The method according to (3), wherein the inducible promoter is T5 promoter, T7 promoter, lacT5 promoter, lacT7 promoter, tac promoter, araBAD promoter, rhaBAD promoter, tet promoter, penP promoter, cspA promoter, or a promoter comprising, as an operator sequence, tetO or lacO operator.
(5) The method according to (4), wherein the inducible promoter is T5 promoter, T7 promoter, lacT5 promoter, lacT7 promoter, or tac promoter.
(6) The method according to (5), wherein the inducible promoter is T5 promoter.
(7) The method according to any one of claims (1) to (6), wherein the gram-negative bacterium is a transformed enteric bacterium.
(8) The method according to any one of (1) to (6), wherein the gram-negative bacterium is a transformed Escherichia coli strain.
(9) A microorganism comprising disruptions of the gene [1] and the gene [2] below and exhibiting enhanced expression levels of the genes [3] or the gene [4] below:
   [1] a gene encoding γ-glutamyltransferase (EC: 2.3.2.2);
   [2] a gene encoding glutathione reductase (EC: 1.8.1.7);
   [3] a gene encoding glutamate-cysteine ligase (EC: 6.3.2.2) and a gene encoding glutathione synthetase (EC: 6.3.2.3); and
   [4] a gene encoding bifunctional glutathione synthetase.
(10) The microorganism according to (9), comprising a disruption of the gene [5] below:
   [5] a gene encoding tripeptide peptidase (EC: 3.4.11.4).
(11) The microorganism according to (9), wherein the microorganism is a transformed bacterium.
(12) The microorganism according to (9), wherein the microorganism is a transformed enteric bacterium.
(13) The microorganism according to (9), wherein the microorganism is a transformed Gram-negative bacterium.
(14) The microorganism according to (9), wherein the microorganism is a transformed E. coli strain.
(15) A method for producing glutathione comprising culturing the microorganism according to (9) in a medium.

### EMBODIMENTS OF THE INVENTION

Hereafter, preferable embodiments of the first aspect and the second aspect and the third aspect of the present disclosure are described in detail, although the technical scope of the first aspect and the second aspect of the present disclosure are not limited to these embodiments.

### 1. Enzymes

### 1.1. γ-Glutamyltransferase

γ-Glutamyltransferase (EC: 2.3.2.2) is an enzyme that hydrolyzes γ-glutamylpeptide, such as glutathione.

“γ-glutamyltransferase” is also referred to as “γ-glutamyl transpeptidase” or “Ggt.” The terms “γ-glutamyltransferase,” “γ-glutamyl transpeptidase,” and “Ggt” are interchangeable herein.

Specific examples of γ-glutamyltransferase include:
(1A) a polypeptide having he amino acid sequence as shown in SEQ ID NO: 22;
(1B) a polypeptide consisting of an amino acid sequence derived from the amino acid sequence as shown in SEQ ID NO: 22 by addition, deletion, or substitution of 1 or a plurality of amino acids (which is a polypeptide consisting of an amino acid sequence derived from the amino acid sequence as shown in SEQ ID NO: 22 preferably by substitution, deletion, and/or addition, and more preferably by deletion and/or addition, of 1 or a plurality of amino acids at either or both of the N terminus and the C terminus) and having γ-glutamyltransferase activity;
(1C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 22 and having γ-glutamyltransferase activity; and
(1D) a fragment of any of the polypeptides (1A) to (1C) having γ-glutamyltransferase activity.

The fragment (1D) can be a polypeptide comprising preferably 200 or more, more preferably 300 or more, more preferably 400 or more, more preferably 500 or more, and more preferably 550 or more amino acids.

The polypeptides may be subjected to adequate chemical modification.

In (1B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution. The term “conservative amino acid substitution” refers to substitution between amino acids having similar properties in terms of, for example, electric charge, side chains, polarity, and aromaticity. Amino acids having similar properties can be classified into: for example, basic amino acids, such as arginine, lysine, and histidine; acidic amino acids, such as aspartic acid and glutamic acid; uncharged polar amino acids, such as glycine, asparagine, glutamine, serine, threonine, cysteine, and tyrosine; nonpolar amino acids, such as leucine, isoleucine, alanine, valine, proline, phenylalanine, tryptophan, and methionine; branched amino acids, such as leucine, valine, and isoleucine; and aromatic amino acids, such as phenylalanine, tyrosine, tryptophan, and histidine.

In (1C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the protein represented by SEQ ID NO: 22. The “sequence identity” can be determined with the use of protein search systems, such as BLAST or FASTA (Karlin, S. et al., 1993, Proc. Natl. Acad. Sci., U.S.A., 90:5873-5877; Altschul, S. F. et al., 1990, J. Mol. Biol., 215:403-410; Pearson, W. R. et al., 1988, Proc. Natl. Acad. Sci., U.S.A., 85:2444-2448). Hereafter, the “sequence identity” of amino acid sequences is used in the same sense.

The term “a gene encoding γ-glutamyltransferase (EC:2.3.2.2)” refers to a gene (a nucleic acid which is DNA or RNA, with DNA being preferable) encoding the amino acid sequence of γ-glutamyltransferase and such gene is included in the genomic DNA in the chromosome of the wild-type microorganism before disruption of γ-glutamyltransferase therein.

SEQ ID NO: 21 shows an example of DNA encoding the amino acid sequence of E. coli-derived γ-glutamyltransferase as shown in SEQ ID NO: 22. It should be noted that the nucleotide sequence as shown in SEQ ID NO: 21 is not always present in that state in the genomic DNA of the wild-type microorganism. The nucleotide sequence as shown in SEQ ID NO: 21 may be an exon sequence comprising one or more intron sequences therein.

Specific examples of nucleotide sequences of genes encoding the amino acid sequence of γ-glutamyltransferase include:
(1E) the nucleotide sequence as shown in SEQ ID NO: 21;
(1F) a nucleotide sequence derived from the nucleotide sequence as shown in SEQ ID NO: 21 by addition, deletion, or substitution of 1 or a plurality of nucleotides (which is preferably a nucleotide sequence derived from the nucleotide sequence as shown in SEQ ID NO: 21 preferably by substitution, deletion, and/or addition, and more preferably by deletion and/or addition, of 1 or a plurality of nucleotides at either or both of the 5′ terminus and the 3′ terminus) and having γ-glutamyltransferase activity;
(1G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 21 and having γ-glutamyltransferase activity;
(1H) a partial nucleotide sequence of any of the nucleotide sequences (1E) to (1G) encoding an amino acid sequence of a polypeptide having γ-glutamyltransferase activity;
(1I) a nucleotide sequence derived from any of the nucleotide sequences (1E) to (1H) by introduction of silent mutation (nucleotide substitution that does not alter amino acids to encode);
(1J) a nucleotide sequence encoding the amino acid sequence of any of the polypeptides (1A) to (1D); and
(1K) a nucleotide sequence comprising, as an exon sequence, any of the nucleotide sequences (1E) to (1J) and one or more intron sequences therein.

In (1G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in SEQ ID NO: 21. The “sequence identity” can be determined with the use of nucleotide sequence search systems, such as BLAST or FASTA (Karlin, S. et al., 1993, Proc. Natl. Acad. Sci., U.S.A., 90:5873-5877; Altschul, S. F. et al., 1990, J. Mol. Biol., 215:403-410; Pearson, W. R. et al., 1988, Proc. Natl. Acad. Sci., U.S.A., 85:2444-2448). Hereafter, the “sequence identity” of nucleotide sequences is used in the same sense.

### 1.2. Glutathione Reductase

Glutathione reductase (EC:1.8.1.7) is an enzyme that catalyzes a reaction of reducing oxidized glutathione (glutathione disulfide) in the presence of NADPH to generate reduced glutathione.

Specific examples of glutathione reductases include:
(2A) a polypeptide consisting of the amino acid sequence as shown in SEQ ID NO: 26;
(2B) a polypeptide consisting of an amino acid sequence derived from the amino acid sequence as shown in SEQ ID NO: 26 by addition, deletion, or substitution of 1 or a plurality of amino acids (which is a polypeptide consisting of an amino acid sequence derived from the amino acid sequence as shown in SEQ ID NO: 26 preferably by substitution, deletion, and/or addition, and more preferably by deletion and/or addition, of 1 or a plurality of amino acids at either or both of the N terminus and the C terminus) and having glutathione reductase activity;
(2C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 26 and having glutathione reductase activity; and
(2D) a fragment of any of the polypeptides (2A) to (2C) having glutathione reductase activity.

The fragment (2D) can be a polypeptide comprising preferably 200 or more, more preferably 300 or more, and more preferably 400 or more amino acids.

In (2B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution. The “conservative amino acid substitution” is as described in (1B) of the <1.1. γ-Glutamyltransferase> section above.

In (2C) above, “sequence identity” is as described in (1C) of the <1.1. γ-Glutamyltransferase> section above. In (2C) above, specifically, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the protein represented by SEQ ID NO: 26.

The term “a gene encoding glutathione reductase (EC:1.8.1.7)” refers to a gene (a nucleic acid which is DNA or RNA, with DNA being preferable) encoding the amino acid sequence of glutathione reductase and such gene is included in the genomic DNA in the chromosome of the wild-type microorganism before disruption of glutathione reductase therein.

SEQ ID NO: 25 shows an example of DNA encoding the amino acid sequence of E. coli-derived glutathione reductase as shown in SEQ ID NO: 26. It should be noted that the nucleotide sequence as shown in SEQ ID NO: 25 is not always present in that state in the genomic DNA of the wild-type microorganism. The nucleotide sequence as shown in SEQ ID NO: 25 may be an exon sequence comprising one or more intron sequences therein.

Specific examples of nucleotide sequences of genes encoding the amino acid sequence of glutathione reductase include:
(2E) the nucleotide sequence as shown in SEQ ID NO: 25;
(2F) a nucleotide sequence derived from the nucleotide sequence as shown in SEQ ID NO: 25 by addition, deletion, or substitution of 1 or a plurality of nucleotides (which is preferably a nucleotide sequence derived from the nucleotide sequence as shown in SEQ ID NO: 25 preferably by substitution, deletion, and/or addition, and more preferably by deletion and/or addition, of 1 or a plurality of nucleotides at either or both of the 5′ terminus and the 3′ terminus) and encoding a polypeptide having glutathione reductase activity;
(2G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 25 and encoding a polypeptide having glutathione reductase activity;
(2H) a partial nucleotide sequence of any of the nucleotide sequences (2E) to (2G) encoding an amino acid sequence of a polypeptide having glutathione reductase activity;
(2I) a nucleotide sequence derived from any of the nucleotide sequences (2E) to (2H) by introduction of silent mutation (nucleotide substitution that does not alter amino acids to encode);
(2J) a nucleotide sequence encoding the amino acid sequence of any of the polypeptides (2A) to (2D); and
(2K) a nucleotide sequence comprising, as an exon sequence, any of the nucleotide sequences (2E) to (2J) and one or more intron sequences therein.

In (2G) above, “sequence identity” is as described in (1G) of the <1.1. γ-Glutamyltransferase> section above. In (2G) above, specifically, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in SEQ ID NO: 25.

### 1.3. Tripeptide Peptidase

Tripeptide peptidase (EC: 3.4.11.4) is an enzyme that catalyzes a reaction of releasing the N-terminal amino acid residue from tripeptide.

“Tripeptide peptidase” is also referred to as “peptidase T” or “PepT.” The terms “tripeptide peptidase,” “peptidase T,” and “PepT” are interchangeable herein.

Specific examples of tripeptide peptidase include:
(5A) a polypeptide consisting of the amino acid sequence as shown in SEQ ID NO: 24;
(5B) a polypeptide consisting of an amino acid sequence derived from the amino acid sequence as shown in SEQ ID NO: 24 by addition, deletion, or substitution of 1 or a plurality of amino acids (which is a polypeptide consisting of an amino acid sequence derived from the amino acid sequence as shown in SEQ ID NO: 24 preferably by substitution, deletion, and/or addition, and more preferably by deletion and/or addition, of 1 or a plurality of amino acids at either or both of the N terminus and the C terminus) and having tripeptide peptidase activity;
(5C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 24 and having tripeptidase activity; and
(5D) a fragment of any of the polypeptides (5A) to (5C) having tripeptidase activity.

The fragment (5D) can be a polypeptide comprising preferably 200 or more, more preferably 300 or more, and more preferably 350 or more amino acids.

The polypeptides may be subjected to adequate chemical modification.

In (5B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution. The “conservative amino acid substitution” is as described in (1B) of the <1.1. γ-Glutamyltransferase> section above.

In (5C) above, “sequence identity” is as described in (1C) of the <1.1. γ-Glutamyltransferase> section above. In (5C) above, specifically, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the protein represented by SEQ ID NO: 24.

The term “a gene encoding tripeptide peptidase (EC: 3.4.11.4)” refers to a gene (a nucleic acid which is (DNA or RNA, with DNA being preferable) encoding the amino acid sequence of tripeptide peptidase and such gene is included in the genomic DNA in the chromosome of the wild-type microorganism before disruption of tripeptide peptidase therein.

SEQ ID NO: 23 shows an example of DNA encoding the amino acid sequence of E. coli-derived tripeptide peptidase as shown in SEQ ID NO: 24. It should be noted that the nucleotide sequence as shown in SEQ ID NO: 23 is not always present in that state in the genomic DNA of the wild-type microorganism. The nucleotide sequence as shown in SEQ ID NO: 23 may be an exon sequence comprising one or more intron sequences therein.

Specific examples of nucleotide sequences of genes encoding the amino acid sequence of tripeptide peptidase include:
(5E) the nucleotide sequence as shown in SEQ ID NO: 23;
(5F) a nucleotide sequence derived from the nucleotide sequence as shown in SEQ ID NO: 23 by addition, deletion, or substitution of 1 or a plurality of nucleotides (which is preferably a nucleotide sequence derived from the nucleotide sequence as shown in SEQ ID NO: 23 preferably by substitution, deletion, and/or addition, and more preferably by deletion and/or addition, of 1 or a plurality of nucleotides at either or both of the 5′ terminus and the 3′ terminus) and encoding a polypeptide having tripeptide peptidase activity;
(5G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 23 and encoding a polypeptide having tripeptide peptidase activity;
(5H) a partial nucleotide sequence of any of the nucleotide sequences (5E) to (5G) encoding an amino acid sequence of a polypeptide having tripeptide peptidase activity;
(5I) a nucleotide sequence derived from any of the nucleotide sequences (5E) to (5H) by introduction of silent mutation (nucleotide substitution that does not alter amino acids to encode);
(5J) a nucleotide sequence encoding the amino acid sequence of any of the polypeptides (5A) to (5D); and
(5K) a nucleotide sequence comprising, as an exon sequence, any of the nucleotide sequences (5E) to (5J) and one or more intron sequences therein.

In (5G) above, “sequence identity” is as described in (1G) of the <1.1. γ-Glutamyltransferase> section above. In (5G) above, specifically, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in SEQ ID NO: 23.

### 1.4. Glutamate-Cysteine Ligase

Glutamate-cysteine ligase (EC: 6.3.2.2) is an enzyme that catalyzes a reaction of recognizing L-cysteine as a substrate in the presence of ATP and allowing L-cysteine to bind to L-glutamic acid to generate γ-glutamylcysteine. Such enzyme is not particularly limited in terms of the origin, the structure, and other properties, provided that it has the activity described above. Such activity is referred to as “glutamate-cysteine ligase activity” herein. At 1 U of the activity, 1 μmol of γ-glutamylcysteine is generated at 30° C. in 1 minute, and such activity is assayed under the conditions described below.

“Glutamate-cysteine ligase” is also referred to as “glutamate cysteine ligase” or “GshA.” The terms “glutamate-cysteine ligase,” “glutamate cysteine ligase,” and “GshA” are interchangeable herein.

### Assay Conditions

An enzyme solution is added to a 50 mM Tris-HCl buffer solution (pH 8.0) containing 10 mM ATP, 15 mM L-glutamic acid, 15 mM L-cysteine, and 10 mM magnesium sulfate, the reaction is allowed to proceed while maintaining the temperature at 30° C., and the reaction is terminated with the addition of 6 N hydrochloric acid. By performing high-performance liquid chromatography, γ-glutamylcysteine in the reaction solution is quantified.

The conditions for the high-performance liquid chromatography are as described below. Under the conditions described below, reduced glutathione (GSH), γ-glutamylcysteine (γ-GC), bis-γ-glutamylcystine (reduced γ-GC), and oxidized glutathione (GSSG) are eluted in that order.

### HPLC Conditions

Column: ODS-HG-3 (4.6 mm q × 150 mm, Nomura Chemical Co., Lid.)
Eluate: A solution prepared by dissolving 12.2 g of potassium dihydrogen-phosphate and 3.6 g of sodium heptane sulfonate in 1.8 1 of distilled water, adjusting pH at 2.8 with the aid of phosphoric acid, and adding 186 ml of methanol
Flow rate: 1.0 ml/min
Column temperature: 40° C.
Assay wavelength: 210 nm

Use of glutamate-cysteine ligase having glutamate-cysteine ligase activity of 0.5 U or higher per 1 mg of a protein (i.e., specific activity) is preferable, with specific activity of 1 U or higher being more preferable, that of 5 U or higher being further preferable, and that of 10 U or higher being the most preferable.

The origin of glutamate-cysteine ligase is not particularly limited, and glutamate-cysteine ligase derived from microorganisms, animals, plants, and the like can be used. Glutamate-cysteine ligase derived from microorganisms is preferable, and glutamate-cysteine ligase derived from enteric bacteria such as Escherichia coli, bacteria such as coryneform bacteria, eukaryotic microorganisms such as yeasts, or the like is particularly preferable.

Specific examples of the nucleotide sequence of E. coli-derived glutamate-cysteine ligase and the amino acid sequence encoded by the nucleotide sequence are shown in SEQ ID NO: 12 and SEQ ID NO: 13, respectively.

Glutamate-cysteine ligase is not limited to the glutamate-cysteine ligase consisting of the amino acid sequence as shown in SEQ ID NO: 13. Other polypeptides having glutamate-cysteine ligase activity, such as active mutants of the glutamate-cysteine ligase or orthologs of different species, may be used. Other polypeptides having glutamate-cysteine ligase activity preferably exhibit activity of 10% or higher, more preferably 40% or higher, more preferably 60% or higher, more preferably 80% or higher, and further preferably 90% or higher, compared with the activity when glutamate-cysteine ligase consisting of the amino acid sequence as shown in SEQ ID NO: 13 is used under the activity assay conditions described above.

Specific examples of glutamate-cysteine ligase include:
(3-1A) a polypeptide consisting of the amino acid sequence as shown in SEQ ID NO: 13;
(3-1B) a polypeptide consisting of an amino acid sequence derived from the amino acid sequence as shown in SEQ ID NO: 13 by addition, deletion, or substitution of 1 or a plurality of amino acids (which is a polypeptide consisting of an amino acid sequence derived from the amino acid sequence as shown in SEQ ID NO: 13 preferably by substitution, deletion, and/or addition, and more preferably by deletion and/or addition, of 1 or a plurality of amino acids at either or both of the N terminus and the C terminus) and having glutamate-cysteine ligase activity;
(3-1C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 13 and having glutamate-cysteine ligase activity; and
(3-1D) a fragment of any of the polypeptides (3-1A) to (3-1C) having glutamate-cysteine ligase activity.

The fragment (3-1D) can be a polypeptide comprising preferably 200 or more, more preferably 300 or more, more preferably 400 or more, more preferably 450 or more, and more preferably 500 or more amino acids.

The polypeptides may be subjected to adequate chemical modification.

In (3-1B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution. The “conservative amino acid substitution” is as described in (1B) of the <1.1. γ-Glutamyltransferase> section above.

In (3-1C) above, “sequence identity” is as described in (1C) of the <1.1. γ-Glutamyltransferase> section above. In (3-1C) above, specifically, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the protein represented by SEQ ID NO: 13.

The term “a gene encoding glutamate-cysteine ligase (EC: 6.3.2.2)” refers to a gene (DNA or RNA, with DNA being preferable) encoding the amino acid sequence of glutamate-cysteine ligase.

SEQ ID NO: 12 shows an example of DNA encoding the amino acid sequence of E. coli-derived glutamate-cysteine ligase as shown in SEQ ID NO: 13. The nucleotide sequence of the gene encoding the amino acid sequence of glutamate-cysteine ligase may be codon-optimized for the host.

Specific examples of nucleotide sequences of genes encoding the amino acid sequence of glutamate-cysteine ligase include:
(3-1E) the nucleotide sequence as shown in SEQ ID NO: 12;
(3-1F) a nucleotide sequence derived from the nucleotide sequence as shown in SEQ ID NO: 12 by addition, deletion, or substitution of 1 or a plurality of nucleotides (which is a nucleotide sequence derived from the nucleotide sequence as shown in SEQ ID NO: 12 preferably by substitution, deletion, and/or addition, and more preferably by deletion and/or addition, of 1 or a plurality of nucleotides at either or both of the 5′ terminus and the 3′ terminus) and encoding a polypeptide having glutamate-cysteine ligase activity;
(3-1G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 12 and encoding a polypeptide having glutamate-cysteine ligase activity;
(3-1H) a partial nucleotide sequence of any of the nucleotide sequences (3-1E) to (3-1G) encoding an amino acid sequence of a polypeptide having glutamate-cysteine ligase activity;
(3-1I) a nucleotide sequence derived from any of the nucleotide sequences (3-1E) to (3-1H) by introduction of silent mutation (nucleotide substitution that does not alter amino acids to encode);
(3-1J) a nucleotide sequence encoding the amino acid sequence of any of the polypeptides (3-1A) to (3-1D); and
(3-1K) a nucleotide sequence comprising, as an exon sequence, any of the nucleotide sequences (3-1E) to (3-1J) and one or more intron sequences therein.

In (3-1G) above, “sequence identity” is as described in (1G) of the <1.1. γ-Glutamyltransferase> section above. In (3-1G) above, specifically, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in SEQ ID NO: 12.

### 1.5. Glutathione Synthetase

Glutathione synthetase (EC: 6.3.2.3) is an enzyme that catalyzes a reaction of recognizing γ-glutamylcysteine as a substrate in the presence of ATP and allowing γ-glutamylcysteine to bind to glycine to generate GSH. Such enzyme is not particularly limited in terms of the origin, the structure, and other properties, provided that it has the activity described above. Such activity is referred to as “glutathione synthetase activity” herein. At 1 U of the activity, 1 μmol of glutathione is generated at 30° C. in 1 minute, and such activity is assayed under the conditions described below. “Glutathione synthetase” is also referred to as “GshB.” The terms “glutathione synthetase” and “GshB” are interchangeable herein.

### Assay Conditions

An enzyme solution is added to a 50 mM Tris-HCl buffer solution (pH 8.0) containing 10 mM ATP, 15 mM γ-glutamylcysteine, 15 mM glycine, and 10 mM magnesium sulfate, the reaction is allowed to proceed while maintaining the temperature at 30° C., and the reaction is terminated with the addition of 6 N hydrochloric acid. By performing high-performance liquid chromatography, glutathione in the reaction solution is quantified.

The conditions for the high-performance liquid chromatography are as described above with regard to the method of glutamate-cysteine ligase activity assay.

Use of glutathione synthetase having glutathione synthetase activity of 0.5 U or higher per 1 mg of a protein (i.e., specific activity) is preferable, with specific activity of 1 U or higher being more preferable, that of 5 U or higher being further preferable, and that of 10 U or higher being the most preferable.

The origin of glutathione synthetase is not particularly limited, and glutathione synthetase derived from microorganisms, animals, plants, and the like can be used. Glutathione synthetase derived from microorganisms is preferable, and glutathione synthetase derived from enteric bacteria such as Escherichia coli, bacteria such as coryneform bacteria, eukaryotic microorganisms such as yeasts, microorganisms of Hydrogenophilales, or the like is particularly preferable.

Glutathione synthetase derived from microorganisms of Hydrogenophilales is preferably glutathione synthetase derived from microorganisms of Thiobacillus, and more preferably glutathione synthetase derived from microorganisms of Thiobacillus denitrificans. Glutathione synthetase derived from the Thiobacillus denitrificans strain ATCC 25259 is particularly preferable.

### Preferable Embodiments of E. coli-Derived Glutathione Synthetase or Mutant Thereof

Specific examples of the nucleotide sequence of E. coli-derived glutathione synthetase and the amino acid sequence encoded by the nucleotide sequence are shown in SEQ ID NO: 14 and SEQ ID NO: 15, respectively.

Glutathione synthetase is not limited to the glutathione synthetase consisting of the amino acid sequence as shown in SEQ ID NO: 15. Other polypeptides having glutathione synthetase activity, such as active mutants of the glutathione synthetase or orthologs of different species, may be used. Other polypeptides having glutathione synthetase activity preferably exhibit activity of 10% or higher, more preferably 40% or higher, more preferably 60% or higher, more preferably 80% or higher, and further preferably 90% or higher, compared with the activity when glutathione synthetase consisting of the amino acid sequence as shown in SEQ ID NO: 15 is used under the activity assay conditions described above.

Specific examples of E. coli-derived glutathione synthetase or a mutant thereof include:
(3-2A) a polypeptide consisting of the amino acid sequence as shown in SEQ ID NO: 15;
(3-2B) a polypeptide consisting of an amino acid sequence derived from the amino acid sequence as shown in SEQ ID NO: 15 by addition, deletion, or substitution of 1 or a plurality of amino acids (which is a polypeptide consisting of an amino acid sequence derived from the amino acid sequence as shown in SEQ ID NO: 15 preferably by substitution, deletion, and/or addition, and more preferably by deletion and/or addition, of 1 or a plurality of amino acids at either or both of the N terminus and the C terminus) and having glutathione synthetase;
(3-2C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 15 and having glutathione synthetase activity; and
(3-2D) a fragment of any of the polypeptides (3-2A) to (3-2C) having glutathione synthetase activity.

The fragment (3-2D) can be a polypeptide comprising preferably 200 or more, more preferably 250 or more, and more preferably 300 or more amino acids.

The polypeptides may be subjected to adequate chemical modification.

In (3-2B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution. The “conservative amino acid substitution” is as described in (1B) of the <1.1. γ-Glutamyltransferase> section above.

In (3-2C) above, “sequence identity” is as described in (1C) of the <1.1. γ-Glutamyltransferase> section above. In (3-2C) above, specifically, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the protein represented by SEQ ID NO: 15.

The term “a gene encoding glutathione synthetase (EC: 6.3.2.3)” refers to a gene (a nucleic acid which is DNA or RNA, with DNA being preferable) encoding the amino acid sequence of glutathione synthetase.

SEQ ID NO: 14 shows an example of DNA encoding the amino acid sequence of E. coli-derived glutathione synthetase as shown in SEQ ID NO: 15. The nucleotide sequence of the gene encoding the amino acid sequence of glutathione synthetase may be codon-optimized for the host.

Specific examples of nucleotide sequences of genes encoding the amino acid sequence of the E. coli-derived glutathione synthetase or a mutant thereof include:
(3-2E) the nucleotide sequence as shown in SEQ ID NO: 14;
(3-2F) a nucleotide sequence derived from the nucleotide sequence as shown in SEQ ID NO: 14 by addition, deletion, or substitution of 1 or a plurality of nucleotides (which is a nucleotide sequence derived from the nucleotide sequence as shown in SEQ ID NO: 14 preferably by substitution, deletion, and/or addition, and more preferably by deletion and/or addition, of 1 or a plurality of nucleotides at either or both of the 5′ terminus and the 3′ terminus) and encoding a polypeptide having glutathione synthetase activity;
(3-2G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 14 and encoding a polypeptide having glutathione synthetase activity;
(3-2H) a partial nucleotide sequence of any of the nucleotide sequences (3-2E) to (3-2G) encoding an amino acid sequence of a polypeptide having glutathione synthetase activity;
(3-2I) a nucleotide sequence derived from any of the nucleotide sequences (3-2E) to (3-2H) by introduction of silent mutation (nucleotide substitution that does not alter amino acids to encode);
(3-2J) a nucleotide sequence encoding the amino acid sequence of any of the polypeptides (3-2A) to (3-2D); and
(3-2K) a nucleotide sequence comprising, as an exon sequence, any of the nucleotide sequences (3-2E) to (3-2J) and one or more intron sequences therein.

In (3-2G) above, “sequence identity” is as described in (1G) of the <1.1. γ-Glutamyltransferase> section above. In (3-2G) above, specifically, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in SEQ ID NO: 14.

### Preferable Embodiments of Thiobacillus denitrificans-Derived Glutathione Synthetase or Mutant Thereof

Other preferable specific examples of glutathione synthetase include wild-type glutathione synthetase derived from the Thiobacillus denitrificans strain ATCC 25259 and active mutants thereof. Specific examples of the nucleotide sequence of the wild-type glutathione synthetase derived from the Thiobacillus denitrificans strain ATCC 25259 and the amino acid sequence encoded by the nucleotide sequence are shown in SEQ ID NO: 16 and SEQ ID NO: 17, respectively. An active mutant of the wild-type glutathione synthetase is a polypeptide preferably exhibiting activity of 10% or higher, more preferably 40% or higher, more preferably 60% or higher, more preferably 80% or higher, and further preferably 90% or higher, compared with the activity when the wild-type glutathione synthetase consisting of the amino acid sequence as shown in SEQ ID NO: 17 is used under the activity assay conditions described above.

Specific examples of glutathione synthetase derived from the Thiobacillus denitrificans strain ATCC 25259 or a mutant thereof include:
(3-3A) a polypeptide consisting of the amino acid sequence as shown in SEQ ID NO: 17;
(3-3B) a polypeptide consisting of an amino acid sequence derived from the amino acid sequence as shown in SEQ ID NO: 17 by addition, deletion, or substitution of 1 or a plurality of amino acids (which is a polypeptide consisting of an amino acid sequence derived from the amino acid sequence as shown in SEQ ID NO: 17 preferably by substitution, deletion, and/or addition, and more preferably by deletion and/or addition, of 1 or a plurality of amino acids at either or both of the N terminus and the C terminus) and having glutathione synthetase activity;
(3-3C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 17 and having glutathione synthetase activity; and
(3-3D) a fragment of any of the polypeptides (3-3A) to (3-3C) having glutathione synthetase activity.

The fragment (3-3D) can be a polypeptide comprising preferably 200 or more, more preferably 250 or more, and more preferably 300 or more amino acids.

The polypeptides may be subjected to adequate chemical modification.

In (3-2B) above, the term “a plurality of” can be, for example, 2 to 20, 2 to 15, 2 to 10, 2 to 7, 2 to 5, 2 to 4, or 2 or 3. Amino acid substitution is preferably conservative amino acid substitution. The “conservative amino acid substitution” is as described in (1B) of the <1.1. γ-Glutamyltransferase> section above.

In (3-3C) above, “sequence identity” is as described in (1C) of the <1.1. γ-Glutamyltransferase>section above. In (3-3C) above, specifically, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the protein represented by SEQ ID NO: 17.

SEQ ID NO: 16 shows an example of DNA encoding the amino acid sequence of glutathione synthetase derived from the Thiobacillus denitrificans strain ATCC 25259 as shown in SEQ ID NO: 17. The nucleotide sequence of the gene encoding the amino acid sequence of glutathione synthetase may be codon-optimized for the host.

Specific examples of nucleotide sequences of genes encoding the amino acid sequence of the glutathione synthetase derived from the Thiobacillus denitrificans strain ATCC 25259 or a mutant thereof include:
(3-3E) the nucleotide sequence as shown in SEQ ID NO: 16;
(3-3F) a nucleotide sequence derived from the nucleotide sequence as shown in SEQ ID NO: 16 by addition, deletion, or substitution of 1 or a plurality of nucleotides (which is preferably a nucleotide sequence derived from the nucleotide sequence as shown in SEQ ID NO: 16 preferably by substitution, deletion, and/or addition, and more preferably by deletion and/or addition, of 1 or a plurality of nucleotides at either or both of the 5′ terminus and the 3′ terminus) and encoding a polypeptide having glutathione synthetase activity;
(3-3G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 16 and encoding a polypeptide having glutathione synthetase activity;
(3-3H) a partial nucleotide sequence of any of the nucleotide sequences (3-2E) to (3-2G) encoding an amino acid sequence of a polypeptide having glutathione synthetase activity;
(3-3I) a nucleotide sequence derived from any of the nucleotide sequences (3-3E) to (3-3H) by introduction of silent mutation (nucleotide substitution that does not alter amino acids to encode);
(3-3J) a nucleotide sequence encoding the amino acid sequence of any of the polypeptides (3-3A) to (3-3D); and
(3-3K) a nucleotide sequence comprising, as an exon sequence, any of the nucleotide sequences (3-3E) to (3-3J) and one or more intron sequences therein.

In (3-3G) above, “sequence identity” is as described in (1G) of the <1.1. γ-Glutamyltransferase> section above. In (3-3G) above, specifically, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in SEQ ID NO: 16.

### Preferable Embodiments of Active Mutants of Thiobacillus denitrificans-Derived Glutathione Synthetase

Another preferable example of glutathione synthetase is an active mutant of wild-type glutathione synthetase derived from the Thiobacillus denitrificans strain ATCC 25259 comprising the amino acid sequence as shown in SEQ ID NO: 17. The polypeptide described in WO 2018/084165 is particularly preferable.

Specific examples of the active mutants include:
(3-4A) a polypeptide consisting of an amino acid sequence 3-4A derived from the amino acid sequence as shown in SEQ ID NO: 17 by substitution of one or a plurality of amino acids selected from the group consisting of amino acids 13, 17, 20, 23, 39, 70, 78, 101, 113, 125, 126, 136, 138, 149, 152, 154, 155, 197, 200, 215, 226, 227, 230, 239, 241, 246, 249, 254, 260, 262, 263, 270, 278, 299, 305, 307, and 310;
(3-4B) a polypeptide consisting of an amino acid sequence derived from the amino acid sequence 3-4A by addition, deletion, or substitution of 1 or a plurality of amino acids other than the amino acids mentioned above (which is a polypeptide consisting of an amino acid sequence derived from the amino acid sequence 3-4A preferably by substitution, deletion, and/or addition, and more preferably by deletion and/or addition, of 1 or a plurality of amino acids at either or both of the N terminus and the C terminus) and having glutathione synthetase activity;
(3-4C) a polypeptide consisting of an amino acid sequence having consistency with the amino acid sequence 3-4A in the amino acid positions mentioned above and 80% or higher, preferably 85% or higher, and more preferably 90% or higher, 95% or higher, 97% or higher, 98% or higher, or 99% or higher sequence identity to the amino acid sequence 3-4A in amino acid positions other than the amino acid positions mentioned above and having glutathione synthetase activity; and
(3-3D) a fragment of any of the polypeptides (3-4A) to (3-4C) having glutathione synthetase activity.

The fragment (3-4D) can be a polypeptide comprising preferably 150 or more, more preferably 200 or more, and more preferably 300 or more amino acids.

The polypeptides may be subjected to adequate chemical modification.

In (3-4B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution. The “conservative amino acid substitution” is as described in (1B) of the <1.1. γ-Glutamyltransferase> section above.

In (3-4C) above, “sequence identity” is as described in (1C) of the <1.1. γ-Glutamyltransferase> section above. In (3-4C) above, specifically, “sequence identity” is a value determined by aligning 2 amino acid sequences, introducing gaps, according to need, so as to maximize the extent of amino acid consistency therebetween, and determining a percentage (%) of identical amino acids based on the total number of amino acid residues other than the amino acid positions mentioned above in the amino acid sequence 3-4A.

It is more preferable that the amino acid sequence 3-4A be an amino acid sequence derived from the amino acid sequence as shown in SEQ ID NO: 17 by one or a plurality of amino acid substitutions selected from the group consisting of: serine at position 13, glutamic acid at position 17, threonine at position 20, leucine at position 23, threonine at position 39, serine at position 70, leucine at position 78, asparagine, glutamine, serine, or threonine at position 101, histidine at position 113, valine at position 125, asparagine at position 126, threonine at position 136, alanine at position 138, glutamine at position 149, glutamine at position 152, asparagine at position 154, leucine at position 155, glutamine at position 197, serine at position 200, aspartic acid at position 215, arginine at position 226, serine at position 227, proline at position 230, serine at position 239, histidine at position 241, arginine at position 246, glutamic acid at position 249, aspartic acid at position 254, alanine, cysteine, glycine, glutamine, or threonine at position 260, cysteine at position 262, arginine at position 263, isoleucine at position 270, glycine or alanine at position 278, alanine at position 299, glycine at position 305, valine at position 307, and threonine at position 310.

It is particularly preferable that the amino acid sequence 3-4A be an amino acid sequence derived from the amino acid sequence as shown in SEQ ID NO: 17 by any of amino acid substitutions (1) to (35) below:
(1) serine at position 13;
(2) glutamic acid at position 17, histidine at position 113, and proline at position 230;
(3) threonine at position 20 and aspartic acid at position 215;
(4) threonine at position 20 and histidine at position 241;
(5) leucine at position 23 and asparagine at position 126;
(6) threonine at position 39 and alanine at position 260;
(7) serine at position 70 and alanine at position 260;
(8) leucine at position 78 and alanine at position 278;
(9) asparagine at position 101;
(10) glutamine at position 101;
(11) serine at position 101;
(12) serine at position 101 and alanine at position 260;
(13) threonine at position 101;
(14) valine at position 125 and glutamic acid at position 249;
(15) valine at position 125 and glutamine at position 152;
(16) threonine at position 136;
(17) alanine at position 138, glutamine at position 149, histidine at position 241, and glutamine at position 263;
(18) asparagine at position 154 and arginine at position 246;
(19) leucine at position 155 and serine at position 239;
(20) glutamine at position 197;
(21) serine at position 200 and alanine at position 260;
(22) arginine at position 226 and alanine at position 260;
(23) serine at position 227 and alanine at position 260;
(24) aspartic acid at position 254 and alanine at position 260;
(25) alanine at position 260;
(26) alanine at position 260, glycine at position 278, and valine at position 307;
(27) alanine at position 260 and alanine at position 299;
(28) alanine at position 260 and glycine at position 305;
(29) alanine at position 260 and threonine at position 310;
(30) cysteine at position 260;
(31) glycine at position 260;
(32) glutamine at position 260;
(33) threonine at position 260;
(34) cysteine at position 262; and
(35) isoleucine at position 270.

The nucleotide sequence encoding the amino acid sequence of any of the polypeptides (3-4A) to (3-4D) above can be used as the “gene encoding glutathione synthetase (EC: 6.3.2.3).”

SEQ ID NO: 18 shows an example of a nucleotide sequence encoding an amino acid sequence of an active mutant resulting from substitution of valine at position 260 with alanine in the amino acid sequence of glutathione synthetase derived from the Thiobacillus denitrificansstrain ATCC 25259 as shown in SEQ ID NO: 17. The nucleotide sequence of the gene encoding the amino acid sequence of the active mutant of glutathione synthetase derived from the Thiobacillus denitrificans strain ATCC 25259 may be codon-optimized for the host.

### 1.6. Bifunctional Glutathione Synthetase

Bifunctional glutathione synthetase is an enzyme that has activity of catalyzing a reaction of recognizing L-cysteine as a substrate in the presence of ATP and allowing L-cysteine to bind to L-glutamic acid to generate γ-glutamylcysteine and activity of catalyzing a reaction of recognizing γ-glutamylcysteine as a substrate in the presence of ATP and allowing γ-glutamylcysteine to bind to glycine to generate GSH. Such enzyme is not particularly limited in terms of the origin, the structure, and other properties, provided that it has the activities described above. Such activities are collectively referred to as “bifunctional glutathione synthetase activity” herein. At 1 U of the activity, 1 μmol of GSH is generated at 30° C. in 1 minute, and such activity is assayed under the conditions described below.

“Bifunctional glutathione synthetase” is also referred to as “GshF.” The terms “bifunctional glutathione synthetase” and “GshF” are interchangeable herein.

### Assay Conditions

An enzyme solution is added to a 50 mM Tris-HCl buffer solution (pH 8.0) containing 10 mM ATP, 15 mM L-glutamic acid, 15 mM L-cysteine, 15 mM glycine, and 10 mM magnesium sulfate, the reaction is allowed to proceed while maintaining the temperature at 30° C., and the reaction is terminated with the addition of 6 N hydrochloric acid. By performing high-performance liquid chromatography, glutathione in the reaction solution is quantified.

The conditions for the high-performance liquid chromatography are as described above with regard to the method of glutamate-cysteine ligase activity assay.

Use of bifunctional glutathione synthetase having bifunctional glutathione synthetase activity of 0.5 U or higher per 1 mg of a protein (i.e., specific activity) is preferable, with specific activity of 1 U or higher being more preferable, that of 5 U or higher being further preferable, and that of 10 U or higher being the most preferable.

The origin of bifunctional glutathione synthetase is not particularly limited, and glutathione synthetase derived from microorganisms, animals, plants, and the like can be used. Bifunctional glutathione synthetase derived from microorganisms is preferable. Bifunctional glutathione synthetase derived from bacteria is particularly preferable. More specifically, bifunctional glutathione synthetase derived from at least one bacterial species selected from the group consisting of the bacteria described below is preferable: bacteria of the genus Streptococcus, such as Streptococcus agalactiae, Streptococcus mutans, Streptococcus suis, and Streptococcus thermophilus; bacteria of the genus Lactobacillus, such as Lactobacillus plantarum; bacteria of the genus Desulfotalea, such as Desulfotalea psychrophile; bacteria of the genus Clostridium, such as Clostridium perfringens; bacteria of the genus Listeria, such as Listeria innocua and Listeria monocytogenes; bacteria of the genus Enterococcus, such as Enterococcus faecalis and Enterococcus faecium; bacteria of the genus Pasteurella, such as Pasteurella multocida; bacteria of the genus Mannheimia, such as Mannheimia succiniciprodecens; and bacteria of the genus Haemophilus, such as Haemophilus somnus.

Specific examples of the nucleotide sequence of the bifunctional glutathione synthetase derived from Streptococcus agalactiae and the amino acid sequence encoded by the nucleotide sequence are shown in SEQ ID NO: 19 and SEQ ID NO: 20, respectively. The nucleotide sequence as shown in SEQ ID NO: 19 encodes the bifunctional glutathione synthetase derived from Streptococcus agalactiae consisting of the amino acid sequence as shown in SEQ ID NO: 20 and exhibits the frequency of codon usage adapted to E. coli.

Bifunctional glutathione synthetase is not limited to the bifunctional glutathione synthetase consisting of the amino acid sequence as shown in SEQ ID NO: 20. Other polypeptides having bifunctional glutathione synthetase activity, such as active mutants of the bifunctional glutathione synthetase or orthologs of different species, may be used. Other polypeptides having bifunctional glutathione synthetase activity preferably exhibit activity of 10% or higher, more preferably 40% or higher, more preferably 60% or higher, more preferably 80% or higher, and further preferably 90% or higher, compared with the activity when the bifunctional glutathione synthetase consisting of the amino acid sequence as shown in SEQ ID NO: 20 is used under the activity assay conditions described above.

Specific examples of bifunctional glutathione synthetase include:
(4A) a polypeptide consisting of the amino acid sequence as shown in SEQ ID NO: 20;
(4B) a polypeptide consisting of an amino acid sequence derived from the amino acid sequence as shown in SEQ ID NO: 20 by addition, deletion, or substitution of 1 or a plurality of amino acids (which is a polypeptide consisting of an amino acid sequence derived from the amino acid sequence as shown in SEQ ID NO: 20 preferably by substitution, deletion, and/or addition, and more preferably by deletion and/or addition, of 1 or a plurality of amino acids at either or both of the N terminus and the C terminus) and having bifunctional glutathione synthetase activity;
(4C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 20 and having bifunctional glutathione synthetase activity; and
(4D) a fragment of any of the polypeptides (4A) to (4C) having glutathione synthetase activity.

The fragment (4D) can be a polypeptide comprising preferably 400 or more, more preferably 500 or more, more preferably 600 or more, more preferably 700 or more, and more preferably 730 or more amino acids.

The polypeptides may be subjected to adequate chemical modification.

In (4B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution. The “conservative amino acid substitution” is as described in (1B) of the <1.1. γ-Glutamyltransferase> section above.

In (4C) above, “sequence identity” is as described in (1C) of the <1.1. γ-Glutamyltransferase> section above. In (4C) above, specifically, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the protein represented by SEQ ID NO: 20.

The term “a gene encoding bifunctional glutathione synthetase” refers to a gene (a nucleic acid which is DNA or RNA, with DNA being preferable) encoding the amino acid sequence of bifunctional glutathione synthetase.

Specific examples of nucleotide sequences of genes encoding the amino acid sequence of the bifunctional glutathione synthetase include:
(4E) the nucleotide sequence as shown in SEQ ID NO: 19;
(4F) a nucleotide sequence derived from the nucleotide sequence as shown in SEQ ID NO: 19 by addition, deletion, or substitution of 1 or a plurality of nucleotides (which is preferably a nucleotide sequence derived from the nucleotide sequence as shown in SEQ ID NO: 19 preferably by substitution, deletion, and/or addition, and more preferably by deletion and/or addition, of 1 or a plurality of nucleotides at either or both of the 5′ terminus and the 3′ terminus) and encoding a polypeptide having bifunctional glutathione synthetase activity;
(4G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 19 and encoding a polypeptide having bifunctional glutathione synthetase activity;
(4H) a partial nucleotide sequence of any of the nucleotide sequences (4E) to (4G) encoding an amino acid sequence of a polypeptide having bifunctional glutathione synthetase activity;
(4I) a nucleotide sequence derived from any of the nucleotide sequences (4E) to (4H) by introduction of silent mutation (nucleotide substitution that does not alter amino acids to encode);
(4J) a nucleotide sequence encoding the amino acid sequence of any of the polypeptides (4A) to (4D); and
(4K) a nucleotide sequence comprising, as an exon sequence, any of the nucleotide sequences (4E) to (4J) and one or more intron sequences therein.

In (4G) above, “sequence identity” is as described in (1G) of the <1.1. γ-Glutamyltransferase> section above. In (4G) above, specifically, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in SEQ ID NO: 19.

### 2. First Aspect of the Present Disclosure

The method for producing γ-glutamylcysteine, bis-γ-glutamylcystine, γ-glutamylcystine, reduced glutathione, and/or oxidized glutathione and the prokaryotic microbial strain according to the first aspect of the present disclosure are described.

### 2.1. Prokaryotic Microbial Strain According to the First Aspect of the Present Disclosure

One or more embodiments of the first aspect of the present disclosure relate to a prokaryotic microbial strain capable of overproduction of γ-glutamylcysteine, bis-γ-glutamylcystine, γ-glutamylcystine, reduced glutathione, and/or oxidized glutathione in which the expression levels of one or more genes selected from genes encoding the glutamate-cysteine ligase, the glutathione synthetase, and the bifunctional glutathione synthetase are increased, compared with the expression levels in the wild-type strain thereof.

The prokaryotic microbial strain according to one or more embodiments of the first aspect of the present disclosure are cultured in a medium, so that they are capable of overproduction of γ-glutamylcysteine, bis-γ-glutamylcystine, γ-glutamylcystine, reduced glutathione, and/or oxidized glutathione and accumulation thereof in the medium. Thus, the prokaryotic microbial strain can be used for efficient production of the peptides. When the prokaryotic microorganism according to one or more embodiments of the first aspect of the present disclosure is cultured in a medium in which the total concentration of cysteine and cystine is 0.5 g/l or lower or in a medium that is not supplemented with cysteine or cystine, the prokaryotic microorganism is capable of production of the peptides. Thus, the peptide production cost can be reduced.

The prokaryotic microbial strain according to one or more embodiments of the first aspect of the present disclosure is preferably capable of overproduction of γ-glutamylcysteine, bis-γ-glutamylcystine, γ-glutamylcystine, reduced glutathione, and/or oxidized glutathione by inducing expression of the one or more genes described above. When the prokaryotic microbial strain according to one or more embodiments of the first aspect of the present disclosure is cultured in a medium and the one or more genes are induced to expressed therein, the prokaryotic microbial strain is capable of overproduction of γ-glutamylcysteine, bis-γ-glutamylcystine, γ-glutamylcystine, reduced glutathione, and/or oxidized glutathione and accumulation thereof in the medium. Thus, the prokaryotic microbial strain can be used for efficient production of the peptides.

Enzymes exhibiting the increased gene expression levels in the prokaryotic microbial strain according to one or more embodiments of the first aspect of the present disclosure may be one or more enzymes selected from among glutamate-cysteine ligase, glutathione synthetase, and bifunctional glutathione synthetase. Specific examples of the enzymes are as described above. When the prokaryotic microbial strain is used for production of γ-glutamylcysteine, bis-γ-glutamylcystine, and/or γ-glutamylcystine, it is preferable that the gene expression levels of the glutamate-cysteine ligase and/or bifunctional glutathione synthetase be increased, compared with the expression levels in the wild-type strain thereof. When the prokaryotic microbial strain is used for production of reduced glutathione and/or oxidized glutathione, it is preferable that the gene expression levels of the glutamate-cysteine ligase and the glutathione synthetase be increased, compared with the expression levels in parent strain or the gene expression levels of the bifunctional glutathione synthetase be increased, compared with the expression levels in the wild-type strain thereof.

In one or more embodiments of the first aspect of the present disclosure, a prokaryotic microbial strain serving as a host is a bacterium. Specific examples thereof include cells of microorganisms of the genera Escherichia, Bacillus, Brevibacterium, and Corynebacterium, with cells of microorganisms of the genus Escherichia being particularly preferable and cells of Escherichia coli being the most preferable. A prokaryotic microbial strain serving as a host may be an enteric bacterium. The prokaryotic microbial strain according to one or more embodiments of the first aspect of the present disclosure can be a transformant of a prokaryotic microorganism carrying a particular gene.

“Wild-type strain” is the host strain before introduction of one or more genes selected from genes encoding the glutamate-cysteine ligase, the glutathione synthetase, and the bifunctional glutathione synthetase. “Wild-type strain” may be referred to as “parent strain.”

The situation in which “the expression levels of one or more genes selected from genes encoding the glutamate-cysteine ligase, the glutathione synthetase, and the bifunctional glutathione synthetase are increased, compared with the expression levels in the wild-type strain thereof” refers to both of the following situations. When the wild-type strain inherently expresses the one or more genes described above, the expression levels of the one or more genes are increased compared with the expression levels thereof in the wild-type strain. When the wild-type strain does not inherently express the one or more genes described above, the capacity for expressing the one or more genes is given to the wild-type strain.

The increased expression levels of the one or more genes can be achieved by increasing the copy number of the one or more genes in the prokaryotic microbial strain or replacing a promoter that regulates the expression of the one or more genes in genomic DNA of the prokaryotic microbial strain with a stronger expression promoter.

The copy number of the one or more genes in cells of a prokaryotic microbial strain can be increased by:
(1) introduction of an expression vector comprising the one or more genes into cells of a prokaryotic microbial strain; or
(2) introduction of the one or more genes into genomic DNA of cells of a prokaryotic microbial strain.

As an expression vector used in the embodiment (1) above, for example, a plasmid vector comprising the one or more genes can be used. It is preferable that an expression vector be capable of autonomous replication in cells of a prokaryotic microbial strain. It is preferable that an expression vector comprise DNA encoding one or more enzymes selected from among glutamate-cysteine ligase, glutathione synthetase, and bifunctional glutathione synthetase and a promoter operably linked to a position where the DNA can be transcribed. It is preferable that an expression vector be capable of autonomous replication in cells of a prokaryotic microbial strain and that an expression vector be recombinant DNA comprising a nucleotide sequence composed of a promoter, a ribosome binding sequence, a nucleotide sequence encoding the amino acid sequences of the one or more enzymes, and a transcription terminator sequence.

Examples of preferable plasmid vectors include: pQEK1, pCA24N (DNA RESEARCH, 12, 191-299, 2005), pACYC177, pACYC184 (available from Nippon Gene Co., Ltd.), pQE30, pQE60, pQE70, pQE80, and pQE9 (available from Qiagen); pTipQC1 (available from Qiagen or Hokkaido System Science Co., Ltd.) and pTipRT2 (available from Hokkaido System Science Co., Ltd.); pBS vector, Phagescript vector, Bluescript vector, pNH8A, pNH16A, pNH18A, and pNH46A (available from Stratagene); ptrc99a, pKK223-3, pKK233-3, pDR540, and pRIT5 (available from Addgene); pRSF (available from MERCK); and pAC (available from Nippon Gene Co., Ltd.), pUCN18 (which can be prepared via modification of pUC18 (available from Takara Bio Inc.)), pSTV28 (available from Takara Bio Inc.), and pUCNT (WO 94/03613).

The expression vector preferably comprises a promoter that regulates transcription of the one or more genes.

In one or more embodiments of the first aspect of the present disclosure, a promoter is preferably an inducible promoter.

Examples of inducible promoters include isopropyl-β-thiogalactopyranoside (IPTG) inducible promoter, photoinducible promoter that induces gene expression under light application, araBAD promoter (arabinose inducible), rhaBAD promoter (rhamnose inducible), tet promoter (drug inducible), penP promoter (drug inducible), cspA promoter (low-temperature inducible promoter), and a promoter comprising, as an operator sequence, tetO or lacO operator, with IPTG inducible promoter, araBAD promoter, rhaBAD promoter, tet promoter, penP promoter, cspA promoter, or a promoter comprising, as an operator sequence, tetO or lacO operator being preferable.

Specific examples of IPTG inducible promoters include T5 promoter, lacUV5 promoter, lac promoter, T7 promoter, lacT5 promoter, lacT7 promoter, and tac promoter. Among various types of inducible promoters, IPTG inducible promoters are particularly preferable. Among IPTG inducible promoters, T5 promoter, T7 promoter, lacT5 promoter, lacT7 promoter, or tac promoter is particularly preferable.

As a promoter, a highly active promoter modified from an existing promoter with the use of various reporter genes can be used. For example, the −35 region and the −10 region in the promoter region may be made close to the consensus sequence so as to enhance promoter activity (WO 2000/018935). Examples of highly active promoters include various tac-like promoters (Katashkina J I et al., Russian Federation patent application Ser. No. 20061,34574). A method for evaluation of promoter strength and examples of strong promoters are described in, for example, the literature of Goldstein et al. (Prokaryotic promoters in biotechnology, Biotechnol. Annu. Rev., 1, 105-128, 1995).

When an expression vector comprising the one or more genes is introduced into cells of a prokaryotic microbial strain, the copy number of the expression vector in the cells is preferably 2 or more, more preferably 3 or more, more preferably 5 or more, more preferably 10 or more, more preferably 15 or more, and more preferably 20 or more.

When the expression levels of two or more genes selected from among the genes are to be increased in cells of a prokaryotic microbial strain, an expression vector may comprise the two or more genes. In such a case, the two or more genes may be positioned under the control of an expression promoter. Alternatively, each of the two or more genes may be included in expression vectors separately from each other.

When the one or more genes are to be introduced into genomic DNA of cells of a prokaryotic microbial strain in accordance with the embodiment (2) above, homologous recombination can be performed.

When a promoter for the one or more genes is to be replaced with a stronger expression promoter in genomic DNA of cells of a prokaryotic microbial strain, a promoter similar to an expression vector can be used as an expression promoter, with an inducible promoter being preferable. Specific examples of preferable promoters are as described above.

In the prokaryotic microbial strain according to one or more embodiments of the first aspect of the present disclosure, an extent of increase in the expression levels of the one or more genes is not particularly limited. The expression levels of the one or more genes can be represented as the amount of mRNAs corresponding to the one or more genes extracted from the cells (i.e., mRNAs encoding the amino acid sequences of one or more enzymes selected from among glutamate-cysteine ligase, glutathione synthetase, and bifunctional glutathione synthetase). Such mRNA-based expression levels are preferably represented relative to the amount of mRNAs encoding adequate internal standard proteins. According to embodiments in which the glutamate-cysteine ligase gene expression level is increased, the expression level of glutamate-cysteine ligase in a prokaryotic microbial strain (preferably, the relative value determined by dividing the amount of mRNA of glutamate-cysteine ligase in a prokaryotic microbial strain by the amount of mRNA encoding the internal standard protein in the same strain) is preferably at least 5 times, more preferably at least 10 times, and further preferably at least 20 times greater than the expression level of glutamate-cysteine ligase in a wild-type strain (preferably, the relative value determined by dividing the amount of mRNA of glutamate-cysteine ligase in a wild-type strain by the amount of mRNA encoding the internal standard protein in the same strain). According to embodiments in which the glutathione synthetase gene expression level is increased, the expression level of glutathione synthetase in a prokaryotic microbial strain (preferably, the relative value determined by dividing the amount of mRNA of glutathione synthetase in a prokaryotic microbial strain by the amount of mRNA encoding the internal standard protein in the same strain) is preferably at least 5 times, more preferably at least 10 times, and further preferably at least 20 times greater than the expression level of glutathione synthetase in a wild-type strain (preferably, the relative value determined by dividing the amount of mRNA of glutathione synthetase in a wild-type strain by the amount of mRNA encoding the internal standard protein in the same strain). An example of the internal standard protein is a protein encoded by hcaT (SEQ ID NO: 27) known as a housekeeping gene.

In the prokaryotic microbial strain according to one or more embodiments of the first aspect of the present disclosure, more preferably, the expression levels of the genes of the enzymes having activity of degrading cysteine, γ-glutamylcysteine, bis-γ-glutamylcystine, γ-glutamylcystine, reduced glutathione, or oxidized glutathione or the glutathione uptake transporter gene are lower than the expression levels thereof in a wild-type strain, or expression of the gene is lost. When culturing the prokaryotic microbial strain, the peptides are likely to be accumulated in the medium.

Examples of enzymes having activity of degrading γ-glutamylcysteine, bis-γ-glutamylcystine, γ-glutamylcystine, reduced glutathione, or oxidized glutathione include γ-glutamyltransferase and tripeptide peptidase. An example of a gene of an enzyme having cysteine-degrading activity is the tryptophanase gene tnaA. An example of a glutathione uptake transporter gene is yliABCD.

Specific examples of γ-glutamyltransferase are as described above.

Specific examples of tripeptide peptidase are as described above.

A prokaryotic microbial strain in which the expression levels of the genes of the enzymes having activity of degrading cysteine, γ-glutamylcysteine, bis-γ-glutamylcystine, γ-glutamylcystine, reduced glutathione, or oxidized glutathione or the glutathione uptake transporter gene are lowered compared with the expression levels thereof in its wild-type strain or the expression of the genes is lost can be produced by a method comprising introducing deletion, substitution, or addition of nucleotides into the nucleotide sequence encoding the enzymes in genomic DNA of the prokaryotic microbial strain. An example of the method is a method involving homologous recombination. Specifically, the method disclosed in JP 2004-344029 A can be employed.

A more preferable embodiment of the prokaryotic microbial strain according to one or more embodiments of the first aspect of the present disclosure relates to a prokaryotic microbial strain carrying one or more genes selected from genes encoding a glutamate-cysteine ligase, a glutathione synthetase, and a bifunctional glutathione synthetase, operably linked to an inducible promoter,
wherein the inducible promoter increases the expression level of the gene encoding glutamate-cysteine ligase gene in the prokaryotic microbial strain by 20 times or greater than that in the wild-type strain thereof, when the one or more genes is the glutamate-cysteine ligase gene, and
the prokaryotic microbial strains are capable of overproducing γ-glutamylcysteine, bis-γ-glutamylcystine, γ-glutamylcystine, reduced glutathione, and/or oxidized glutathione by induced expression of the one or more genes.

In the prokaryotic microbial strain according to the more preferable embodiment described above, the one or more genes operably linked to the inducible promoter may be included in a part of genomic DNA of the prokaryotic microbial strain, or the one or more genes may be included in the expression vector existing in the prokaryotic microbial strain. Specific examples of the expression vectors are as described above.

Inducible promoters are not particularly limited, provided that the promoters increase the expression level of the gene encoding glutamate-cysteine ligase in the prokaryotic microbial strain by 20 times or greater than that in the wild-type strain thereof, when the one or more genes are the genes encoding glutamate-cysteine ligase. The transcription level can be evaluated based on the amount of mRNA. The gene encoding glutamate-cysteine ligase used to determine as to whether or not the inducible promoter has the given expression capacity is preferably of the same type with the gene encoding glutamate-cysteine ligase of the wild-type strain.

Such highly active inducible promoter can be selected from among the examples of inducible promoters above. Preferable examples include IPTG inducible promoter, photoinducible promoter, araBAD promoter, rhaBAD promoter, tet promoter, penP promoter, cspA promoter, and a promoter comprising, as an operator sequence, tetO or lacO operator. IPTG inducible promoter is particularly preferable as a highly active inducible promoter. Among various types of IPTG inducible promoters, T5 promoter, T7 promoter, lacT5 promoter, lacT7 promoter, or tac promoter is particularly preferable.

As a highly active inducible promoter, a highly active inducible promoter modified with the use of various reporter genes as described above can be used.

### 2.2. The Method for Producing a Useful Substance Via Cell Culture According to the First Aspect of the Present Disclosure

Further one or more embodiments of the first aspect of the present disclosure relate to a method for producing γ-glutamylcysteine, bis-γ-glutamylcystine, γ-glutamylcystine, reduced glutathione, and/or oxidized glutathione (hereafter, referred to as a “target peptide”), which comprises culturing a prokaryotic microbial strain in which the expression levels of one or more genes selected from genes encoding a glutamate-cysteine ligase, a glutathione synthetase, and a bifunctional glutathione synthetase are increased, compared with the expression levels thereof in its wild-type strain, in a medium in which the total concentration of cysteine and cystine is 0.5 g/l or lower.

The prokaryotic microbial strain used in the method is preferably capable of overproducing the target peptides by induced expression of the one or more genes.

Further one or more embodiments of the first aspect of the present disclosure relates to a method for producing the target peptides, wherein the method comprises culturing a prokaryotic microbial strain in which the expression levels of one or more genes selected from genes encoding a glutamate-cysteine ligase, a glutathione synthetase, and a bifunctional glutathione synthetase are increased, compared with the expression levels thereof in its wild-type strain, in a medium, and wherein the method does not comprises adding cysteine or cystine to the medium.

The prokaryotic microbial strain used in the method is preferably capable of overproducing the target peptides by induced expression of the one or more genes.

The methods are based on the unpredictable finding, such that the prokaryotic microbial strain according to one or more embodiments of the first aspect of the present disclosure is capable of accumulating the target peptides comprising, as a constituent amino acid, cysteine or cystine in a medium even when culture is performed in a medium in which the total concentration of cysteine and cystine is as low as 0.5 g/l or lower or a medium that is not supplemented with cysteine or cystine. According to the methods, the target peptides can be produced at low cost.

In the methods described above, either a synthetic or natural medium may be used, provided that it contains nutrients necessary for the growth of microorganisms used in the first aspect of the present disclosure, such as carbon sources, nitrogen sources, inorganic salts, and vitamins, and for the biosynthesis of the target peptides.

Any carbon sources can be used, provided that carbon sources are assimilated by microorganisms used. Examples of carbon sources include saccharides, such as glucose and fructose, alcohols, such as ethanol and glycerol, and organic acids, such as acetic acid.

Examples of nitrogen sources include nitrogen compounds, such as ammonia, ammonium salt such as ammonium sulfate, and amine, and natural nitrogen sources, such as peptone and soybean hydrolysate.

Examples of inorganic salts include potassium phosphate, magnesium sulfate, sodium chloride, ferrous sulfate, and potassium carbonate.

Examples of vitamins include biotin and thiamine. According to need, substances required for the growth of the microorganisms according to the first aspect of the present disclosure (e.g., auxotrophic amino acids in the case of amino acid auxotrophic microorganisms) can be added.

The medium is supplemented with at least one of a sulfur source and glycine and it is preferably supplemented with both of the sulfur source and glycine. For example, glycine can be added to a medium at 100 mM to 2000 mM, and preferably 400 mM to 1200 mM. The sulfur source can be added to the medium at, for example, 100 mM to 2000 mM, and preferably 400 mM to 1200 mM.

As sulfur sources, one or more types of inorganic sulfur compounds, such as sulfuric acid, thiosulfuric acid, sulfurous acid, hyposulfite, sulfide, or a salt thereof can be added. Sulfuric acid, thiosulfuric acid, sulfurous acid, hyposulfite, or sulfide may be in a free form, salt, or a compound of any thereof. Examples of salts include, but are not particularly limited to, sodium salt, calcium salt, and ammonium salt, and potassium salt.

Glycine may be in a free form, salt, or a compound of any thereof. Examples of salts include, but are not particularly limited to, sulfate and hydrochloride.

Sulfur sources and/or glycine can be added to a medium when the culture is initiated or during culture. Sulfur sources and/or glycine may be added to the medium simultaneously, continuously, or intermittently.

Sulfur sources and/or glycine may be contained in the medium throughout the culture period or in a part of the culture period. For example, it is not necessary that the amount of sulfur sources and glycine to be added is within the range described above throughout the stage at which the target peptides are produced and accumulated. Sulfur sources and/or glycine may be added to the medium in a manner such that the content would be within the range described above during culture. With the elapse of time, the content of sulfur sources and/or glycine may be lowered. Sulfur sources and/or glycine may further be added continuously or intermittently. The concentration of medium components other than sulfur sources and/or glycine may vary during culture, and such medium components may further be added.

Culture is preferably carried out under aerobic conditions, such as via shake culture or aeration-agitation culture. Culture is performed at 20° C. to 50° C., preferably at 20° C. to 42° C., and more preferably at 28° C. to 38° C. At the time of culture, a pH is 5 to 9, and preferably 6 to 7.5. A culture period is 3 hours to 5 days, and preferably 5 hours to 3 days.

The target peptides accumulated in the culture product can be collected in accordance with a conventional purification method. After the completion of culture, for example, bacteria or solids are removed from the culture product via centrifugation, and the target peptides can be collected via ion exchange, concentration, or crystal fractionation.

### 3. The Second Aspect of the Present Disclosure

The microorganism and the method for producing glutathione according to the second aspect of the present disclosure are described.

The term “glutathione” used herein may refer to reduced glutathione, oxidized glutathione, or a mixture of reduced glutathione and oxidized glutathione. The terms “glutathione” and “reduced glutathione and/or oxidized glutathione” are interchangeable herein.

### 3.1. Host Microorganisms

Microorganisms serving as host strains (parent strains) of the microorganisms according to one or more embodiments of the second aspect of the present disclosure in which the gene [1] and the gene [2] below are disrupted and in which expression of the gene [3] or the gene [4] is enhanced are preferably bacteria. The bacteria may be enteric bacteria. The bacteria may be Gram-negative bacteria, such as bacteria of the genus Escherichia or bacteria of the genus Pantoea, or Gram-positive bacteria, such as bacteria of the genus Bacillus, bacteria of the genus Brevibacterium, or bacteria of the genus Corynebacterium, with Gram-negative bacteria being preferable and E. coli being particularly preferable.

The microorganism according to one or more embodiments of the second aspect of the present disclosure can be a transformant comprising disruptions of particular genes and operably carrying particular genes derived from an existing microorganism.

### 3.2. Microorganism According to the Second Aspect of the Present Disclosure

One or more embodiments of the second aspect of the present disclosure relate to a microorganism comprising disruptions of the gene [1] and the gene [2] and exhibiting enhanced expression of the gene [3] or the gene [4]:
[1] a gene encoding γ-glutamyltransferase (EC: 2.3.2.2);
[2] a gene encoding glutathione reductase (EC: 1.8.1.7);
[3] a gene encoding glutamate-cysteine ligase (EC: 6.3.2.2) and a gene encoding glutathione synthetase (EC: 6.3.2.3); and
[4] a gene encoding bifunctional glutathione synthetase.

The microorganism yields high glutathione productivity by fermentation and thus is suitable for glutathione production. When the microorganism is cultured in a medium, it can produce glutathione.

The microorganism more preferably comprises a disruption of the gene [5] below:
[5] a gene encoding tripeptide peptidase (EC: 3.4.11.4).

The microorganism further comprising a disruption of the tripeptide peptidase gene yield particularly high glutathione productivity and are thus preferable.

Microorganisms serving as hosts of the microorganism according to one or more embodiments of the second aspect of the present disclosure are as described above.

Disruption of particular genes in the microorganism according to one or more embodiments of the second aspect of the present disclosure is described.

When the gene encoding γ-glutamyltransferase, the gene encoding glutathione reductase, or the gene encoding tripeptide peptidase (hereafter, such gene may be referred to as a “gene to be disrupted”) is “disrupted” in the microorganism according to one or more embodiments of the second aspect of the present disclosure, activity of an enzyme encoded by the gene to be disrupted is lowered, compared with the activity of the parent strain, and activity is completely quenched. The microorganism according to one or more embodiments of the second aspect of the present disclosure is deprived of functions of the gene to be disrupted, or such functions are lowered in the microorganism. In such microorganism, specifically, the levels of mRNA, which is a transcription product of the gene to be disrupted, or a protein, which is a translation product thereof, are lowered, or mRNA, which is a transcription product of the gene to be disrupted, or a protein, which is a translation product thereof, would not normally function as mRNA or a protein.

Disruption of the gene to be disrupted can be achieved by, for example, artificial modification of the gene of the microbial parent strain. Such modification can be achieved by, for example, mutagenesis, gene recombination, gene expression control using RNAi, or gene editing.

Mutagenesis can be performed via ultraviolet application or via treatment using a common agent causing mutation, such as N-methyl-N′-nitro-N-nitrosoguanidine (MNNG), ethyl methanesulfonate (EMS), or methyl methanesulfonate (MMS).

Gene recombination can be performed in accordance with a known technique (e.g., FEMS Microbiology Letters 165, 1998, 335-340, JOURNAL OF BACTERIOLOGY, December 1995, pp. 7171-7177, Curr. Genet., 1986,10 (8): pp. 573-578, or WO 98/14600).

A gene encoding γ-glutamyltransferase, glutathione reductase, or tripeptide peptidase indicates, in addition to a coding region of the amino acid sequence of each protein, its expression control sequence (e.g., a promoter sequence), an exon sequence, an intron sequence, or the like without distinguishing them from each other. When an expression control sequence is to be modified, preferably one or more nucleotides, more preferably two or more nucleotides, and particularly preferably 3 or more nucleotides in the expression control sequence are modified.

In the second aspect of the present disclosure, disruption of a gene is more preferably disruption of the gene in the genomic DNA of the microorganism. The disruption of the gene may be disruption of a part or the whole of the expression control sequence or disruption of a part or the whole of the coding region of the amino acid sequence of the enzyme. The term “disruption” used herein refers to deletion or damage, with “deletion” being preferable.

The entire gene, including upstream and downstream sequences of the gene to be disrupted, may be deleted in the genomic DNA of the microbial parent strain. When a part or the whole of the coding region of the amino acid sequence of the enzyme encoded by the gene to be disrupted is to be deleted, either of the N-terminal region, the internal region, the C-terminal region, and other regions may be deleted, provided that enzyme activity can be lowered. In general, the gene can be inactivated with certainty by deletion of a longer region. The reading frames of the upstream and downstream sequences of the region to be deleted are preferably inconsistent. A preferable embodiment is directed to a microorganism comprising deletion of a region consisting of a number of nucleotides that is preferably at least 50%, more preferably at least 60%, more preferably at least 70%, more preferably at least 80%, more preferably at least 90%, and more preferably 100% of the total number of nucleotides constituting at least a part of the coding region of the amino acid sequence and/or the expression control sequence of the gene to be disrupted of genomic DNA, such as the coding region and/or the expression control sequence. It is particularly preferable that the microorganism comprises disruption of a region from the start codon to the stop codon of the gene to be disrupted in genomic DNA.

Other examples of disruption of the gene to be disrupted to lower the enzyme activity include damage of the gene, such as introduction of amino acid substitution (missense mutation), introduction of a stop codon (nonsense mutation), and introduction of frameshift mutation via addition or deletion of 1 or 2 nucleotides into the amino acid sequence coding region of the gene to be disrupted in genomic DNA.

Disruption of the gene to be disrupted to lower the enzyme activity can be achieved by, for example, insertion of another sequence into the expression control sequence or the amino acid sequence coding region of the gene to be disrupted in genomic DNA. While another sequence may be inserted into any region of the gene, the gene can be inactivated with certainty via insertion of a longer sequence. It is preferable that reading frames of upstream and downstream sequences of the site of insertion be inconsistent. While “another sequence” is not particularly limited as long as functions of the protein to be encoded are lowered or quenched, examples thereof include a marker gene and a gene useful for production of a γ-glutamyl compound, such as glutathione.

Disruption of the gene to be disrupted in genomic DNA can be achieved by, for example, preparing an inactive gene by modifying the gene to be disrupted so as not to produce a protein that normally functions, transforming microorganisms with recombinant DNA containing the inactive gene, and causing homologous recombination between the inactive gene and a gene in genomic DNA to substitute the gene in genomic DNA with the inactive gene. In such a case, a marker gene may be incorporated into recombinant DNA in accordance with traits of hosts, such as auxotrophic properties. Thus, a procedure of interest is easily performed. The recombinant DNA may be linearized via cleavage with restriction enzymes, so that a strain comprising recombinant DNA integrated into genomic DNA can be efficiently obtained. If a protein encoded by the inactive gene is generated, a conformation thereof would be different from that of a wild-type protein, and functions thereof would be lowered or quenched.

For example, microorganisms may be transformed with linear DNA comprising an arbitrary sequence and, at both ends of the arbitrary sequence, upstream and downstream sequences of the target site of mutation (typically a part of or the entire gene to be disrupted) in genomic DNA or linear DNA comprising upstream and downstream sequences of the target site of mutation in genomic DNA directly ligated to each other to cause homologous recombination in regions upstream and downstream of the target site of mutation in genomic DNA of the microorganisms. Thus, the target site of mutation can be substituted with the sequence of the linear DNA in a single step. The arbitrary sequence may comprise, for example, a marker gene sequence. A marker gene may be removed later, according to need. When a marker gene is to be removed, sequences for homologous recombination may be added to both ends of the marker gene, so as to efficiently remove the marker gene.

Whether or not the gene to be disrupted has been disrupted in the microorganism can be examined based on a lowering in the activity of the enzyme encoded by the gene to be disrupted. The lowering in enzyme activity can be verified by assaying the enzyme activity. For example, glutathione reductase activity can be assayed in accordance with a conventional technique (e.g., the Glutathione Reductase Assay Kit, 7510-100-K, Cosmo Bio Co., Ltd.).

A lowering in the transcription level of the gene to be disrupted can be verified by comparing the amount of mRNA transcribed from the gene of interest with the amount of mRNA of the parent strain. The amount of mRNA can be evaluated by, for example, Northern hybridization or RT-PCR (e.g., Molecular cloning, Cold Spring Harbor Laboratory Press, Cold spring Harbor, U.S.A., 2001). It is preferable that the amount of mRNA be lowered to, for example, 50% or lower, 20% or lower, 10% or lower, 5% or lower, or 0% of the amount of mRNA of the parent strain.

A lowering in the amount of an enzyme encoded by the gene to be disrupted can be verified via Western blotting using an antibody (Molecular cloning, Cold Spring Harbor Laboratory Press, Cold spring Harbor, U.S.A., 2001). Concerning the microorganism according to one or more embodiments of the second aspect of the present disclosure, the amounts of enzymes encoded by the gene to be disrupted are preferably lowered to, for example, 50% or lower, 20% or lower, 10% or lower, 5% or lower, or 0% of the parent strain.

Subsequently, expression enhancement of a particular gene in the microorganism according to one or more embodiments of the second aspect of the present disclosure is described.

Microorganisms in which “the expression of the particular expression-enhanced genes (i.e., genes encoding a glutamate-cysteine ligase, a glutathione synthetase or a bifunctional glutathione synthetase) are enhanced” satisfy both the following conditions. That is, when the parent strain (wild-type strain) of the microorganisms inherently expresses the expression-enhanced genes, the expression levels of the expression-enhanced genes are increased compared with those of the parent strains. When the parent strains do not inherently express the expression-enhanced genes, also, the parent strains are provided with the capacity of expressing the expression-enhanced genes.

The expression levels of the expression-enhanced genes can be increased by increasing the copy number of the expression-enhanced genes in the microbial cells or substituting a promoter that controls the expression-enhanced gene expression in genomic DNA of the microbial cells with a stronger expression promoter.

The copy number of the expression-enhanced genes in the microbial cells can be increased via:
(A) introduction of an expression vector comprising the expression-enhanced genes into microbial cells; or
(B) introduction of the expression-enhanced genes into genomic DNA of microbial cells.

In the embodiment (A) above, for example, a plasmid vector comprising the expression-enhanced genes can be used as an expression vector. It is preferable that an expression vector be capable of autonomous replication in microbial cells. It is preferable that an expression vector comprise DNA encoding one or more enzymes selected from among glutamate-cysteine ligase, glutathione synthetase, and bifunctional glutathione synthetase and a promoter operably linked to a position where the DNA can be transcribed. It is preferable that an expression vector be capable of autonomous replication in microbial cells and that an expression vector be recombinant DNA comprising a nucleotide sequence composed of a promoter, a ribosome binding sequence, a nucleotide sequence encoding the amino acid sequences of the one or more enzymes, and a transcription terminator sequence.

The microorganism according to one or more embodiments of the second aspect of the present disclosure preferably carries an expression vector comprising the nucleotide sequences encoding the expression-enhanced genes. The microorganism is capable of expression of the expression-enhanced genes from the expression vector.

Examples of preferable plasmid vectors include: pQEK1, pCA24N (DNA RESEARCH, 12, 191-299, 2005), pACYC177, pACYC184 (available from Nippon Gene Co., Ltd.), pQE30, pQE60, pQE70, pQE80, and pQE9 (available from Qiagen); pTipQC1 (available from Qiagen or Hokkaido System Science Co., Ltd.) and pTipRT2 (available from Hokkaido System Science Co., Ltd.); pBS vector, Phagescript vector, Bluescript vector, pNH8A, pNH16A, pNH18A, and pNH46A (available from Stratagene); ptrc99a, pKK223-3, pKK233-3, pDR540, and pRIT5 (available from Addgene); pRSF (available from MERCK); and pAC (available from Nippon Gene Co., Ltd.), pUCN18 (which can be prepared via modification of pUC18 (available from Takara Bio Inc.)), pSTV28 (available from Takara Bio Inc.), and pUCNT (WO 94/03613).

The expression vector preferably comprises a promoter that regulates transcription of the expression-enhanced genes.

In one or more embodiments of the second aspect of the present disclosure, a promoter is preferably an inducible promoter.

Examples of inducible promoters include isopropyl-β-thiogalactopyranoside (IPTG) inducible promoter, photoinducible promoter that induces gene expression under light application, araBAD promoter (arabinose inducible), rhaBAD promoter (rhamnose inducible), tet promoter (drug inducible), penP promoter (drug inducible), cspA promoter (low-temperature inducible promoter), and a promoter comprising, as an operator sequence, tetO or lacO operator, with IPTG inducible promoter, araBAD promoter, rhaBAD promoter, tet promoter, penP promoter, cspA promoter, or a promoter comprising, as an operator sequence, tetO or lacO operator being preferable.

Specific examples of IPTG inducible promoters include T5 promoter, lacUV5 promoter, lac promoter, T7 promoter, lacT5 promoter, lacT7 promoter, and tac promoter. Among various types of inducible promoters, IPTG inducible promoters are particularly preferable. Among IPTG inducible promoters, T5 promoter, T7 promoter, lacT5 promoter, lacT7 promoter, or tac promoter is particularly preferable.

As a promoter, a highly active promoter modified from an existing promoter with the use of various reporter genes can be used. For example, the −35 region and the −10 region in the promoter region may be made close to the consensus sequence so as to enhance promoter activity (WO 2000/018935). Examples of highly active promoters include various tac-like promoters (Katashkina J I et al., Russian Federation patent application Ser. No. 20/061,34574). A method for evaluation of promoter strength and examples of strong promoters are described in, for example, the literature of Goldstein et al. (Prokaryotic promoters in biotechnology, Biotechnol. Annu. Rev., 1, 105-128, 1995).

When an expression vector comprising the expression-enhanced genes is introduced into microbial cells, the copy number of the expression vector in the cells is preferably 2 or more, more preferably 3 or more, more preferably 5 or more, more preferably 10 or more, more preferably 15 or more, and more preferably 20 or more.

When the expression levels of the two or more expression-enhanced genes are to be increased in microbial cells, an expression vector may comprise the two or more genes. In such a case, the two or more genes may be positioned under the control of an expression promoter. Alternatively, each of the two or more genes may be included in expression vectors separately from each other.

When the expression-enhanced genes are to be introduced into genomic DNA of microbial cells in accordance with the embodiment (B) above, homologous recombination can be performed.

When a promoter for the expression-enhanced genes is to be replaced with a stronger expression promoter in genomic DNA of microbial cells, a promoter similar to an expression vector can be used as an expression promoter, with an inducible promoter being preferable. Specific examples of preferable promoters are as described above.

In the microorganism according to one or more embodiments of the second aspect of the present disclosure, an extent of expression enhancement of the expression-enhanced genes (i.e., increase in the expression levels) is not particularly limited. The expression levels of the expression-enhanced genes can be represented as the amounts of mRNAs corresponding to the expression-enhanced genes extracted from the cells (i.e., mRNAs encoding the amino acid sequences of one or more enzymes selected from among glutamate-cysteine ligase, glutathione synthetase, and bifunctional glutathione synthetase). Such mRNA-based expression levels are preferably represented relative to the amounts of mRNAs encoding adequate internal standard proteins. According to one or more embodiments of microorganisms in which the expression level of the gene encoding glutamate-cysteine ligase is enhanced, the expression level of the gene encoding glutamate-cysteine ligase in microorganisms (preferably, the relative value determined by dividing the amount of mRNA encoding the glutamate-cysteine ligase in a microbial strain by the amount of mRNA encoding the internal standard protein in the same strain) is preferably at least 5 times, more preferably at least 10 times, and further preferably at least 20 times greater than the expression level of glutamate-cysteine ligase in a parent strain (preferably, the relative value determined by dividing the amount of mRNA encoding the glutamate-cysteine ligase in a wild-type strain by the amount of mRNA encoding the internal standard protein in the same strain). According to one or more embodiments of microorganisms in which the expression of the gene encoding glutathione synthetase is enhanced, the expression level of the gene encoding glutathione synthetase in microorganisms (preferably, the relative value determined by dividing the amount of mRNA encoding glutathione synthetase in a microbial strain by the amount of mRNA encoding the internal standard protein in the same strain) is preferably at least 5 times, more preferably at least 10 times, and further preferably at least 20 times greater than the expression level of glutathione synthetase in a wild-type strain (preferably, the relative value determined by dividing the amount of mRNA of glutathione synthetase in a wild-type strain by the amount of mRNA encoding the internal standard protein in the same strain). An example of the internal standard protein is a protein encoded by the hcaT gene known as a housekeeping gene.

### 3.3. Method for Producing Glutathione According to the Second Aspect of the Present Disclosure

Further one or more embodiments of the second aspect of the present disclosure relate to a method for producing glutathione, which comprises culturing the microorganism according to one or more embodiments of the second aspect of the present disclosure in a medium.

The method for producing glutathione according to the present embodiment are capable of efficient production of glutathione.

The medium may be either a synthetic or natural medium, provided that it contains nutrients necessary for the growth of the microorganism used in the second aspect of the present disclosure, such as carbon sources, nitrogen sources, inorganic salts, and vitamins, and for the biosynthesis of glutathione. Use of M9 medium is preferable.

Any carbon sources can be used, provided that carbon sources are assimilated by the microorganism used. Examples of carbon sources include saccharides, such as glucose and fructose, alcohols, such as ethanol and glycerol, and organic acids, such as acetic acid.

Examples of nitrogen sources include nitrogen compounds, such as ammonia, ammonium salt such as ammonium sulfate, and amine, and natural nitrogen sources, such as peptone and soybean hydrolysate.

Examples of inorganic salts include potassium phosphate, magnesium sulfate, sodium chloride, ferrous sulfate, and potassium carbonate.

Examples of vitamins include biotin and thiamine. According to need, substances required for the growth of the microorganism according to one or more embodiments of the second aspect of the present disclosure (e.g., auxotrophic amino acids in the case of amino acid auxotrophic microorganism) can be added.

The medium is supplemented with at least one of a sulfur source and glycine and it is preferably supplemented with both of the sulfur source and glycine. For example, glycine can be added to a medium at 100 mM to 2000 mM, and preferably 400 mM to 1200 mM. The sulfur source can be added to the medium at, for example, 100 mM to 2000 mM, and preferably 400 mM to 1200 mM.

As sulfur sources, one or more types of inorganic sulfur compounds, such as sulfuric acid, thiosulfuric acid, sulfurous acid, hyposulfite, sulfide, or a salt thereof can be added. Sulfuric acid, thiosulfuric acid, sulfurous acid, hyposulfite, or sulfide may be in a free form, salt, or a compound of any thereof. Examples of salts include, but are not particularly limited to, sodium salt, calcium salt, ammonium salt, and potassium salt.

Glycine may be in a free form, salt, or a compound of any thereof. Examples of salts include, but are not particularly limited to, sulfate and hydrochloride.

Sulfur sources and/or glycine can be added to a medium when the culture is initiated or during culture. Sulfur sources and/or glycine may be added to the medium simultaneously, continuously, or intermittently.

Sulfur sources and/or glycine may be contained in the medium throughout the culture period or in a part of the culture period. For example, it is not necessary that the amount of sulfur sources and glycine to be added is within the range described above throughout the stage at which the target peptides are produced and accumulated. Sulfur sources and/or glycine may be added to the medium in a manner such that the content would be within the range described above during culture. With the elapse of time, the content of sulfur sources and/or glycine may be lowered. Sulfur sources and/or glycine may further be added continuously or intermittently. The concentration of medium components other than sulfur sources and/or glycine may vary during culture, and such medium components may further be added.

Culture is preferably carried out under aerobic conditions, such as via shake culture or aeration-agitation culture. Culture is performed at 20° C. to 50° C., preferably at 20° C. to 42° C., and more preferably at 28° C. to 38° C. At the time of culture, a pH is 5 to 9, and preferably 6 to 7.5. A culture period is 3 hours to 5 days, and preferably 5 hours to 3 days.

Glutathione accumulated in the culture product can be collected in accordance with a conventional purification method. After the completion of culture, for example, bacteria or solids are removed from the culture product via centrifugation, and glutathione can be collected via ion exchange, concentration, or crystal fractionation.

### EXAMPLES

Hereafter, the first aspect and the second aspect of the present disclosure are described in greater detail with reference to the examples, although the first aspect and the second aspect of the present disclosure are not limited to these examples.

Genetic engineering described below can be performed with reference to Molecular Cloning (Cold Spring Harbor Laboratory Press, 1989). Enzymes, cloning hosts, and materials used for genetic engineering may be purchased from commercial providers and used in accordance with the instructions. The enzymes are not particularly limited, provided that they can be used for genetic engineering.

### The First Aspect of the Present Disclosure

The results of experimentation demonstrating the first aspect of the present disclosure are provided below.

### Example 1-1

### Construction of Glutathione Synthetic Gene Expression Vector (1)

The T5 promoter, the E. coli-derived gshA gene (SEQ ID NO: 12), and the E. coli-derived gshB gene (SEQ ID NO: 14) were inserted into a space between the SmaI site and the HindIII site of the plasmid vector pQEKI-term as shown in SEQ ID NO: 4. Primers were designed in accordance with the instructions of the NEBuilder HiFi DNA Assembly Master Mix (New England Biolabs), and the vector was constructed in accordance with the designated procedure.

### Example 1-2

### Construction of Glutathione synthetic gene expression vector (2)

The T5 promoter, the E. coli-derived gshA gene (SEQ ID NO: 12), and the TDgshB (V260A) gene (SEQ ID NO: 18) encoding the mutant enzyme (WO 2018/084165) of glutathione synthetase derived from sulfur bacteria Thiobacillus denitrificans were inserted into a space between the SmaI site and the HindIII site of the plasmid vector pQEK1-term as shown in SEQ ID NO: 4. Primers were designed in accordance with the instructions of the NEBuilder HiFi DNA Assembly Master Mix (New England Biolabs), and the vector was constructed in accordance with the designated procedure. The plasmid vector constructed is designated to be “pQEK1-PT5-ABTd(V260A)-term.”

### Example 1-3

### Construction of Glutathione Synthetic Gene Expression Vector (3)

The T5 promoter and the SA gshF gene (SEQ ID NO: 19) encoding the bifunctional glutathione synthetase gene derived from Streptococcus agalactiae were inserted into a space between the SmaI site and the HindIII site of the plasmid vector pQEK1-term as shown in SEQ ID NO: 4. Primers were designed in accordance with the instructions of the NEBuilder HiFi DNA Assembly Master Mix (New England Biolabs), and the vector was constructed in accordance with the designated procedure. The plasmid vector constructed is designated to be “pQEK1-PT5-FSa-term.”

### Example 1-4

### Construction of Glutathione Synthetic Gene Expression Vector (4)

The lac promoter, the E. coli-derived gshA gene (SEQ ID NO: 12), and the TDgshB (V260A) gene (SEQ ID NO: 18) encoding the mutant enzyme (WO 2018/084165) of glutathione synthetase derived from sulfur bacteria Thiobacillus denitrificans were inserted into a space between the SmaI site and the HindIII site of the plasmid vector pQEK1-term as shown in SEQ ID NO: 4. Primers were designed in accordance with the instructions of the NEBuilder HiFi DNA Assembly Master Mix (New England Biolabs), and the vector was constructed in accordance with the designated procedure. The plasmid vector constructed is designated to be “pQEK1-Plac-ABTd(V260A)-term.”

### Example 1-5

### Construction of Glutathione Synthetic Gene Expression Vector (5)

The lacUV5 promoter, the E. coli-derived gshA gene (SEQ ID NO: 12), and the TDgshB (V260A) gene (SEQ ID NO: 18) encoding the mutant enzyme (WO 2018/084165) of glutathione synthetase derived from sulfur bacteria Thiobacillus denitrificans were inserted into a space between the SmaI site and the HindIII site of the plasmid vector pQEK1-term as shown in SEQ ID NO: 4. Primers were designed in accordance with the instructions of the NEBuilder HiFi DNA Assembly Master Mix (New England Biolabs), and the vector was constructed in accordance with the designated procedure. The plasmid vector constructed is designated to be “pQEK1-PlacUV5-ABTd(V260A)-term.”

### Example 1-6

### Preparation of Host Strains

The E. coli strain BW25113 obtained from the National Institute of Genetics (Japan) was subjected to a treatment using the plasmid pTH18cs1 obtained from the National Institute of Genetics in accordance with the method of preparing a cytosine deaminase-deficient strain disclosed in JP 2004-344029 A to prepare strains comprising disruptions of the γ-glutamyltransferase gene (SEQ ID NO: 21) and the tripeptide peptidase gene (SEQ ID NO: 23).

### Example 1-7

### Preparation of Glutathione Synthetic Gene Expression-Enhanced Strains

Competent cells of the host cells prepared in Example 1-6 were prepared in accordance with a conventional technique and transformed with the plasmid vectors prepared in Example 1-1 to Example 1-5. Thus, transformants were obtained.

### Example 1-8

### Evaluation of Production of Glutathione by Fermentation

The host strains prepared in Example 1-6 (without plasmid) or the glutathione synthetic gene expression-enhanced strains prepared in Example 1-7 were inoculated into 5 ml of LB medium containing 20 μg/ml tetracycline and shake-cultured at 300 rpm and 30° C. for 8 hours. The culture solution (1 ml) was inoculated into 100 ml of M9 medium (6 g/l disodium hydrogen-phosphate, 3 g/l potassium dihydrogen-phosphate, 0.5 g/l sodium chloride, 1 g/l ammonium chloride, 1 mM magnesium sulfate, 0.001% thiamine-hydrochloric acid, 0.1 mM calcium chloride, 2% glucose) supplemented with 20 μg/ml tetracycline. After inoculation, the culture solution was cultured using a culture apparatus (Bio Jr.8, Able Corporation) at 34° C. and pH 6.5 with shaking at 1,000 rpm and aeration of 100 ml/min for 18 hours. The culture solution 18 hours after the initiation of culture (20 ml) was inoculated into 2 liters of M9 medium supplemented with 20 μg/ml tetracycline. After the second inoculation, the culture solution was cultured using a culture apparatus (Bioneer-Neo, Marubishi Bioengineering Co., Ltd.) at 34° C. and pH 6.7 with shaking at 600 rpm and aeration of 4 l/min. During culture, a 50 w/v % glucose solution was added, according to need, so as to maintain the glucose concentration to 15 g/l or higher in the system. 0.1 mM isopropyl-β-thiogalactopyranoside was added 6 hours after the initiation of culture, and, at the same time, 780 mM glycine and 780 mM sodium sulfate were added. An adequate amount of the culture solution was sampled 24 hours after the initiation of culture, and cells were separated from the supernatant via centrifugation. The supernatant was adequately diluted with distilled water, GSH and GSSG in the culture supernatant were quantified by the method described in WO 2016/002884, and the total concentration was determined. The results of quantification of the total concentration of GSH and GSSG in the culture supernatant are shown in Table 1. The culture solution used in the above experiment does not substantially contain cysteine or cystine and the total concentration thereof is less than 0.5 g/l therein.

TABLE 1

| | Total amount of GSH + GSH accumulated in supernatant (g/l) |
|---|---|
| Host strain (without plasmid) | 0.2 |
| pQEK1-PT5-ABTd(V260A)-term | 5.8 |
| pQEK1-Plac-ABTd(V260A)-term | 0.9 |
| pQEK1-PlacUV5-ABTd(V260A)-term | 1.3 |
| pQEK1-PT5-ABEc-term | 5.9 |
| pQEK1-PT5-FSa-term | 4.7 |

### Example 1-9

### Transcription Analysis of the Glutamate-Cysteine Ligase Gene and the Glutathione Synthetase Gene

The expression levels of the overexpressed genes were analyzed using plasmids via real-time PCR. The culture solution after the second inoculation in the culture described in Example 1-8 was cultured for 6 hours, 0.1 mM isopropyl-β-thiogalactopyranoside was added thereto, and an adequate amount of the sample was obtained 1 hour later. RNA was extracted using NucleoSpin RNA purchased from Takara Bio Inc. in accordance with the instructions. RNA samples were diluted with water to adjust the concentration to 50 ng/μl. Reverse transcription was performed using the PrimeScript RT reagent Kit (Perfect Real Time) purchased from Takara Bio Inc. in accordance with the instructions and cDNA was synthesized from RNA. With the use of TB Green Premix Ex Taq II (Tli RNaseH Plus) purchased from Takara Bio Inc. and the QuantStudio 3 real-time PCR system (Thermo Fisher Scientific), gshA, TDgshB (V260A), gshB, and SAgshF in the samples were quantified. As the internal standard, hcaT (SEQ ID NO: 27) known as a housekeeping gene was used. hcaT, gshA, and gshB were subjected to real-time PCR simultaneously with the samples using genomic DNA of E. coli host strains, and calibration curves were prepared. On the basis of the calibration curves, the amounts of genes contained in the cDNAs were quantified. The calibration curve of TDgshB (V260A) was prepared using the pTDGSH2m15 plasmid described in WO 2018/084165, and the calibration curve of SAgshF was prepared using the pNGSHF plasmid described in WO 2016/017631. The quantified value of each gene in the same sample was divided by the quantified value of the internal standard hcaT, and the standardized value was designated to be the expression level of each gene. The forward primer shown in SEQ ID NO: 28 and the reverse primer shown in SEQ ID NO: 29 were used for hcaT amplification. The forward primer shown in SEQ ID NO: 30 and the reverse primer shown in SEQ ID NO: 31 were used for gshA amplification. The forward primer shown in SEQ ID NO: 32 and the reverse primer shown in SEQ ID NO: 33 were used for TDgshB (V260A) amplification. The forward primer shown in SEQ ID NO: 34 and the reverse primer shown in SEQ ID NO: 35 were used for gshB amplification. The forward primer shown in SEQ ID NO: 36 and the reverse primer shown in SEQ ID NO: 37 were used for SAgshF amplification.

The expression level of the E. coli-derived gshA gene in a transformant into which a plasmid vector comprising the E. coli-derived gshA gene; i.e., pQEK1-PT5-ABTd(V260A)-term, pQEK1-Plac-ABTd(V260A)-term, pQEK1-PlacUV5-ABTd(V260A)-term, or pQEK1-PT5-ABEc-term, had been introduced (i.e., the expression level standardized by dividing the quantified gshA gene expression level in a transformant by the quantified hcaT gene expression level in the same transformant) was determined as a relative value base on the E. coli-derived gshA gene expression level in a untransformed host strain prepared in Example 1-6 (i.e., the expression level standardized by dividing the quantified gshA gene expression level in a host strain by the quantified hcaT gene expression level in the same host strain) designated to be 1. The relative value of 5 to less than 10 was evaluated “+,” that of 10 to less than 20 was evaluated “++,” and that of 20 or more was evaluated “+++.” Also, the expression level of the E. coli-derived gshB gene in a transformant into which a plasmid vector comprising the E. coli-derived gshB gene; i.e., pQEK1-PT5-ABEc-term, had been introduced (i.e., the expression level standardized by dividing the quantified gshB gene expression level in a transformant by the quantified hcaT gene expression level in the same transformant) was determined as a relative value base on the E. coli-derived gshB gene expression level in a untransformed host strain (i.e., the expression level standardized by dividing the quantified gshB gene expression level in a host strain by the quantified hcaT gene expression level in the same host strain) designated to be 1. The relative value of 5 to less than 10 was evaluated “+,” that of 10 to less than 20 was evaluated “++,” and that of 20 or more was evaluated “+++.” The results are shown in Table 2.

TABLE 2

| | E. coli gshA | E. coli gshB |
|---|---|---|
| pQEK1-PT5-ABTd(V260A)-term | +++ | − |
| pQEK1-Plac-ABTd(V260A)-term | + | − |
| pQEK1-PlacUV5-ABTd(V260A)-term | ++ | − |
| pQEK1-PT5-ABEc-term | +++ | +++ |

Relative to the gene expression level in the untransformed host strain, 1
+: 5 to less than 10 of the host strain
++: 10 to less than 20 of the host strain
+++: 20 or more of the host strain

Concerning the TDgshB (V260A) or SAgshF gene expression level in a transformant into which a plasmid vector comprising the TDgshB (V260A) or SAgshF gene that is not inherent to the E. coli host strain; i.e., pQEK1-PT5-ABTd(V260A)-term, pQEK1-Plac-ABTd(V260A)-term, pQEK1-PlacUV5-ABTd(V260A)-term, or pQEK1-PT5-FSa-term, had been introduced, the quantified gene expression level was divided by the quantified hcaT gene expression level in the same sample to obtain a standardized value. As a negative control, the gene expression level in the untransformed host strain (without plasmid) was determined. The results are shown in Table 3.

TABLE 3

| | TDgshB | |
|---|---|---|
| | (V260A) | SAgshF |
| Host strain (without plasmid) | 0 | 0 |
| pQEK1-PT5-ABTd(V260A)-term | 1.39 | |
| pQEK1-Plac-ABTd(V260A)-term | 0.08 | |
| pQEK1-PlacUV5-ABTd(V260A)-term | 0.13 | |
| pQEK1-PT5-FSa-term | | 12.44 |

Relative to the hcaT gene expression level in the same sample, 1

### The Second Aspect of the Present Disclosure

Subsequently, the results of experimentation demonstrating the second aspect of the present disclosure are provided below.

### Analysis of Glutathione Concentration in Culture Solution

The glutathione concentration in the culture solution was determined by high-performance liquid chromatography (HPLC, Shimadzu Corporation).

HPLC conditions are as described below.
Column: Develosil ODS-HG-3 4.6 mm×250 mm (Nomura Chemical Co., Ltd.)
Mobile phase: A solution of 30.5 g of potassium dihydrogen-phosphate and 18 g of sodium heptane sulfonate in 4.5 liters of distilled water was prepared, a pH of the solution was adjusted to 3 with phosphoric acid, 250 ml of methanol was added thereto, and a pH of the solution was readjusted to 3 with phosphoric acid.
Flow rate: 1 ml/min
Detection: UV detector, λ=210 nm
Column temperature: 40° C.
Amount of injection: 10 μl

When analyzing the glutathione concentration in the culture solution, cells were removed via centrifugation, and the supernatant was allowed to pass through a syringe filter (q=0.2 μm, Advantech Co., Ltd.) to obtain a culture supernatant. The culture supernatant was diluted to 10-fold with distilled water and the resultant was then subjected to HPLC.

### Production Example 2-1

### Preparation of BW25113Δggt Strain

At the outset, a plasmid vector for disrupting the ggt (γ-glutamyltransferase) gene (SEQ ID NO: 21) was prepared. A DNA fragment comprising the upstream sequence and the downstream sequence of the ggt gene (SEQ ID NO: 1) was obtained by PCR using synthetic oligo DNA. The resulting fragment was digested with XbaI and HindIII, the temperature-sensitive plasmid pTH18cs1 (GenBank Accession Number: AB019610, Hashimoto-Gotoh, T., Gene, 241, 185-191, 2000) was digested with XbaI and HindIII, and the digested fragments were ligated to each other with the aid of Ligation high Ver. 2 (Toyobo Co., Ltd.) to obtain the plasmid vector, pTH18cs1-ggt-UD.

Subsequently, the BW25113Δggt strain was prepared using pTH18cs1-ggt-UD. pTH18cs1-ggt-UD was introduced into the E. coli BW25113 strain via electroporation, applied to an LB agar plate containing chloramphenicol at 10 μg/ml, and cultured at 30° C. to obtain transformants. The resulting transformants were shake-cultured in an LB liquid medium containing chloramphenicol at 10 μg/ml at 30° C. overnight, the culture solution was applied to an LB agar plate containing chloramphenicol at 10 μg/ml, and culture was performed at 42° C. to obtain transformants. The resulting transformants were cultured in an LB liquid medium at 42° C. overnight and applied to an LB agar plate to obtain colonies. The resulting colonies were replica-plated to an LB agar plate and an LB agar plate containing chloramphenicol at 10 μg/ml, and chloramphenicol-sensitive transformants were selected. The selected transformants were analyzed by PCR and using a DNA sequencer to isolate a strain having deletion of a region from the start codon to the stop codon of the ggt gene on the chromosome. This gene-disrupted strain was designated to be the BW25113Δggt strain.

The BW25113Δggt strain is derived from the E. coli BW25113 host strain, and it has deletion of a region from the start codon to the stop codon of the ggt gene on the chromosome.

### Production Example 2-2

### Preparation of BW25113ΔggtΔpepT Strain

At the outset, a plasmid vector for disrupting the pepT (tripeptide peptidase) gene (SEQ ID NO: 23) was prepared. A DNA fragment comprising the upstream sequence and the downstream sequence of the pepT gene (SEQ ID NO: 2) was obtained by PCR using synthetic oligo DNA. The resulting fragment was digested with XbaI and HindIII, pTH18cs1 was digested with XbaI and HindIII, and the digested fragments were ligated to each other with the aid of Ligation high Ver. 2 to obtain the plasmid vector, pTH18cs1-pepT-UD.

Subsequently, a strain derived from a parent strain; i.e., the BW25113Δggt strain prepared in Production Example 2-1, and having deletion of a region from the start codon to the stop codon of the pepT gene on the chromosome was isolated using pTH18cs1-pepT-UD in the same manner as in Production Example 2-1. This gene-disrupted strain was designated to be the BW25113ΔggtΔpepT strain.

The BW25113ΔggtΔpepT strain is derived from the E. coli BW25113 host strain, and it has deletion of a region from the start codon to the stop codon of the ggt gene and that of the pepT gene on the chromosome.

### Production Example 2-3

### Preparation of pQEK1-PT5-ABTd (V260A)-Term

At the outset, the pQEK1 vector as shown in SEQ ID NO: 3 was constructed from pQE-80L (QIAGEN) by replacing the drug-resistant marker with a tetracycline-resistant gene, so as to construct a vector for introducing a gene into E. coli. In addition, a lambda phage-derived terminator sequence was inserted into the HindIII locus of pQEK1 to construct the pQEK1-term vector as shown in SEQ ID NO: 4.

Subsequently, a DNA fragment comprising the T5 promoter, the E. coli-derived gshA gene, and the Thiobacilus denitrificans-derived gshB gene (with V260A mutation) (SEQ ID NO: 5) was obtained by PCR using synthetic oligo DNA. The resulting fragment was ligated to a fragment obtained by digesting pQEK1-term with SpeI and HindIII using NEBuilder HiFi DNA Assembly Master Mix (New England Biolabs) to obtain pQEK1-PT5-ABTd(V260A)-term shown in SEQ ID NO: 6.

### Production Example 2-4

### Preparation of BW25113ΔggtΔpepT/pQEK1-PT5-ABTd (V260A)-Term Strain

pQEK1-PT5-ABTd (V260A)-term prepared in Production Example 2-3 was introduced into the BW25113ΔggtΔpepT strain prepared in Production Example 2-2 via electroporation, and the resultant was applied to an LB agar plate containing tetracycline at 20 μg/ml to select transformants. The selected transformants were subjected to PCR analysis to isolate a strain comprising pQEK1-PT5-ABTd (V260A)-term introduced thereinto. This strain was designated to be the BW25113ΔggtΔpepT/pQEK1-PT5-ABTd(V260A)-term strain.

### Production Example 2-5

### Preparation of BW25113ΔggtΔpepTAgor Strain

At the outset, a plasmid vector for disrupting the gor (glutathione reductase) gene was prepared. A DNA fragment comprising the upstream sequence and the downstream sequence of the gor gene (SEQ ID NO: 7) was obtained by PCR using synthetic oligo DNA. The resulting fragment was digested with XbaI and HindIII, pTH18cs1 was digested with XbaI and HindIII, and the digested fragments were ligated to each other with the aid of Ligation high Ver. 2 to obtain the plasmid vector, pTH18cs1-gor-UD.

Subsequently, a strain derived from a parent strain; i.e., the BW25113ΔggtΔpepT strain prepared in Production Example 2-2, and having deletion of a region from the start codon to the stop codon of the gor gene on the chromosome was isolated using pTH18cs1-gor-UD in the same manner as in Production Example 2-1. This gene-disrupted strain was designated to be the BW25113ΔggtΔpepTΔgor strain.

### Production Example 2-6

### Preparation of BW25113ΔggtΔpepTΔgor/pQEK1-PT5-ABTd(V260A)-Term Strain

pQEK1-PT5-ABTd (V260A)-term prepared in Production Example 2-3 was introduced into the BW25113 ΔggtΔpepTΔgor strain prepared in Production Example 2-5 via electroporation, and the resultant was applied to an LB agar plate containing tetracycline at 20 μg/ml to select transformants. The selected transformants were subjected to PCR analysis to isolate a strain comprising pQEK1-PT5-ABTd(V260A)-term introduced thereinto. This strain was designated to be the BW25113ΔggtΔpepTΔgor/pQEK1-PT5-ABTd(V260A)-term strain.

### Production Example 2-7

### Preparation of pQEK1-PT5-ABEc-Term

A DNA fragment comprising the T5 promoter, the E. coli-derived gshA gene, and the E. coli-derived gshB gene (SEQ ID NO: 8) was obtained by PCR using synthetic oligo DNA. The resulting fragment was ligated to a fragment obtained by digesting pQEK1-term with SpeI and HindIII using NEBuilder HiFi DNA Assembly Master Mix to obtain pQEK1-PT5-ABEc-term shown in SEQ ID NO: 9.

### Production Example 2-8

### Preparation of BW25113ΔggtΔpepT/pQEK1-PT5-ABEc-Term Strain

pQEK1-PT5-ABEc-term prepared in Production Example 2-7 was introduced into the BW25113ΔggtΔpepT strain prepared in Production Example 2-2 via electroporation, and the resultant was applied to an LB agar plate containing tetracycline at 20 μg/ml to select transformants. The selected transformants were subjected to PCR analysis to isolate a strain comprising pQEK1-PT5-ABEc-term introduced thereinto. This strain was designated to be the BW25113ΔggtΔpepT/pQEK1-PT5-ABEc-term strain.

### Production Example 2-9

### Preparation of BW25113ΔggtΔpepTΔgor/pQEK1-PT5-ABEc-Term Strain

pQEK1-PT5-ABEc-term prepared in Production Example 2-3 was introduced into the BW25113ΔggtΔpepTΔgor strain prepared in Production Example 2-5 via electroporation, and the resultant was applied to an LB agar plate containing tetracycline at 20 μg/ml to select transformants. The selected transformants were subjected to PCR analysis to isolate a strain comprising pQEK1-PT5-ABEc-term introduced thereinto. This strain was designated to be the BW25113ΔggtΔpepTΔgor/pQEK1-PT5-ABEc-term strain.

### Production Example 2-10

### Preparation of pQEK1-PT5-FSa-Term

A DNA fragment comprising the T5 promoter and the Streptococcus agalactiae-derived gshF gene (SEQ ID NO: 10) was obtained by PCR using synthetic oligo DNA. The resulting fragment was ligated to a fragment obtained by digesting pQEK1-term with SpeI and HindIII using NEBuilder HiFi DNA Assembly Master Mix to obtain pQEK1-PT5-FSa-term shown in SEQ ID NO: 11.

### Production Example 2-11

### Preparation of BW25113ΔggtΔpepT/pQEK1-PT5-FSa-Term Strain

pQEK1-PT5-FSa-term prepared in Production Example 2-10 was introduced into the BW25113ΔggtΔpepT strain prepared in Production Example 2-2 via electroporation, and the resultant was applied to an LB agar plate containing tetracycline at 20 μg/ml to select transformants. The selected transformants were subjected to PCR analysis to isolate a strain comprising pQEK1-PT5-FSa-term introduced thereinto. This strain was designated to be the BW25113ΔggtΔpepT/pQEK1-PT5-FSa-term strain.

### Production Example 2-12

### Preparation of BW25113ΔggtΔpepTΔgor/pQEK1-PT5-FSa-Term Strain

pQEK1-PT5-FSa-term prepared in Production Example 2-10 was introduced into the BW25113ΔggtΔpepTΔgor strain prepared in Production Example 2-5 via electroporation, and the resultant was applied to an LB agar plate containing tetracycline at 20 g/ml to select transformants. The selected transformants were subjected to PCR analysis to isolate a strain comprising pQEK1-PT5-FSa-term introduced thereinto. This strain was designated to be the BW25113ΔggtΔpepTΔgor/pQEK1-PT5-FSa-term strain.

### Example 2-1

### Production of Glutathione By Fermentation Using The BW25113ΔggtΔpepTΔgor/pQEK1-PT5-ABTd (V260A)-Term Strain

The BW25113ΔggtΔpepTΔgor/pQEK1-PT5-ABTd (V260A)-term strain obtained in Production Example 2-6 was cultured under the conditions described below to produce GSH and GSSG. The resultant was inoculated into 5 ml of LB medium containing 20 ug/ml tetracycline and shake-cultured therein at 300 rpm and 30° C. for 8 hours. The culture solution (1 ml) was inoculated into 100 ml of M9 medium (6 g/l disodium hydrogen-phosphate, 3 g/l potassium dihydrogen-phosphate, 0.5 g/l sodium chloride, 1 g/l ammonium chloride, 1 mM magnesium sulfate, 0.001% thiamine-hydrochloric acid, 0.1 mM calcium chloride, 2% glucose) supplemented with 20 μg/ml tetracycline. After inoculation, the culture solution was cultured using a culture apparatus (Bio Jr.8, Able Corporation) at 34° C. and pH 6.5 with shaking at 1,000 rpm and aeration of 100 ml/min for 18 hours. The culture solution 18 hours after the initiation of culture (20 ml) was inoculated into 2 liters of M9 medium supplemented with 20 ug/ml tetracycline and then cultured using a culture apparatus (Bioneer-Neo, Marubishi Bioengineering Co., Ltd.) at 34° C. and pH 6.7 with shaking at 600 rpm and aeration of 4 l/min. During culture, a 50 w/v % glucose solution was added, according to need, so as to maintain the glucose concentration to 15 g/l or higher in the system. 0.1 mM isopropyl-β-thiogalactopyranoside was added 6 hours after the initiation of culture, and, at the same time, glycine and sodium sulfate were added to adjust the final concentration to 100 mM. An adequate amount of the culture solution was sampled 30 hours after the initiation of culture, and cells were separated from the supernatant via centrifugation. The supernatant was adequately diluted with distilled water, and GSH and GSSG were quantified by HPLC analysis. The results of quantification are shown in Table 4.

### Comparative Example 2-1

### Production of Glutathione by Fermentation Using the BW25113ΔggtΔpepT/pQEK1-PT5-ABTd (V260A)-Term Strain

The BW25113ΔggtΔpepT/pQEK1-PT5-ABTd (V260A)-term strain obtained in Production Example 2-4 was cultured under the same conditions as those in Example 2-1 to produce GSH and GSSG. The results are shown in Table 4.

TABLE 4

| | | GSH + |
|---|---|---|
| | Strains | GSSG (g/l) |
| Ex. 2-1 | BW25113 ΔggtΔpepTΔgor/ | 8.1 |
| | pQEK1-PT5-ABTd*-term | |
| Comp. | BW25113 ΔggtΔpepT/ | 6.8 |
| Ex. 2-1 | pQEK1-PT5-ABTd*-term | |

### Examination

The results of Example 2-1 and the results of Comparative Example 2-1 shown in Table 4 demonstrate that glutathione productivity (GSH+GSSG) is increased to a significant extent by disruption of the gor gene. This indicates that disruption of the gor gene is effective for glutathione production by fermentation.

### Example 2-2

### Production of Glutathione by Fermentation Using the BW25113ΔggtΔpepTΔgor/pQEK1-PT5-ABEc-Term Strain

The BW25113ΔggtΔpepTΔgor/pQEK1-PT5-ABEc-term strain obtained in Production Example 2-9 was cultured under the same conditions as those in Example 2-1 to produce GSH and GSSG. The results are shown in Table 5.

### Comparative Example 2-2

### Production of Glutathione by Fermentation Using the BW25113ΔggtΔpepT/pQEK1-PT5-ABEc-Term Strain

The BW25113ΔggtΔpepT/pQEK1-PT5-ABEc-term strain obtained in Production Example 2-8 was cultured under the same conditions as those in Example 2-1 to produce GSH and GSSG. The results are shown in Table 5.

TABLE 5

| | | GSH + |
|---|---|---|
| | Strains | GSSG (g/l) |
| Ex. 2-2 | BW25113 ΔggtΔpepTΔgor/ | 8.5 |
| | pQEK1-PT5-ABEc-term | |
| Comp. | BW25113 ΔggtΔpepT/ | 7.0 |
| Ex. 2-2 | pQEK1-PT5-ABEc-term | |

### Examination

The results of Example 2-2 and the results of Comparative Example 2-2 shown in Table 5 demonstrate that glutathione productivity (GSH+GSSG) is increased to a significant extent by disruption of the gor gene. This indicates that disruption of the gor gene is effective for glutathione production by fermentation.

### Example 2-3

### Production of Glutathione by Fermentation Using the BW25113ΔggtΔpepTΔgor/pQEK1-PT5-FSa-Term Strain

The BW25113ΔggtΔpepTΔgor/pQEK1-PT5-FSa-term strain obtained in Production Example 2-12 was cultured under the same conditions as those in Example 2-1 to produce GSH and GSSG. The results are shown in Table 6.

### Comparative Example 2-3

### Production of Glutathione by Fermentation Using the BW25113ΔggtΔpepT/pQEK1-PT5-FSa-Term Strain

The BW25113ΔggtΔpepT/pQEK1-PT5-FSa-term strain obtained in Production Example 2-11 was cultured under the same conditions as those in Example 2-1 to produce GSH and GSSG. The results are shown in Table 6.

TABLE 6

| | | GSH + |
|---|---|---|
| | Strains | GSSG (g/l) |
| Ex. 2-3 | BW25113 ΔggtΔpepTΔgor/ | 6.6 |
| | pQEK1-PT5-FSa-term | |
| Comp. | BW25113 ΔggtΔpepT/ | 5.0 |
| Ex. 2-3 | pQEK1-PT5-FSa-term | |

### Examination

The results of Example 2-3 and the results of Comparative Example 2-3 shown in Table 6 demonstrate that glutathione productivity (GSH+GSSG) is increased to a significant extent by disruption of the gor gene. This indicates that disruption of the gor gene is effective for glutathione production by fermentation.

All publications, patents, and patent applications cited herein are incorporated herein by reference in their entirety.

1. A microorganism comprising disruptions of the gene [1] and the gene [2] below and exhibiting enhanced expression levels of the genes [3] or the gene [4] below:
[1] a gene encoding γ-glutamyltransferase (EC: 2.3.2.2);
[2] a gene encoding glutathione reductase (EC:1.8.1.7);
[3] a gene encoding glutamate-cysteine ligase (EC:6.3.2.2) and a gene encoding glutathione synthetase (EC:6.3.2.3); and
[4] a gene encoding bifunctional glutathione synthetase,
wherein the microorganism is a transformed Escherichia coli strain.

2. The microorganism according to claim 1, wherein one or more of said genes [3] or said gene [4] is operably linked to an inducible promoter,
wherein, when the one or more of said genes [3] or said gene [4] is the gene encoding glutamate-cysteine ligase operably linked to the inducible promoter, the inducible promoter increases the expression level of the gene encoding glutamate-cysteine ligase in the microorganism by at least 20 times greater than that of the wild-type strain thereof.

3. The microorganism according to claim 2, wherein the inducible promoter is IPTG inducible promoter, photoinducible promoter, araBAD promoter, rhaBAD promoter, tet promoter, penP promoter, cspA promoter, or a promoter comprising, as an operator sequence, tetO or lacO operator.

4. The microorganism according to claim 3, wherein the inducible promoter is T5 promoter, T7 promoter, lacT5 promoter, lacT7 promoter, tac promoter, araBAD promoter, rhaBAD promoter, tet promoter, penP promoter, cspA promoter, or a promoter comprising, as an operator sequence, tetO or lacO operator.

5. The microorganism according to claim 4, wherein the inducible promoter is T5 promoter, T7 promoter, lacT5 promoter, lacT7 promoter, or tac promoter.

6. The microorganism according to claim 5. wherein the inducible promoter is T5 promoter.

7. The microorganism according to claim 1. comprising a disruption of the gene [5] [5] a gene encoding tripeptide peptidase (EC: 3.4.11.4). below:
